(19) United States
(12) Patent Application Publication
(10) Pub. No.: US 2023/0256055 A1
(43) Pub. Date: Aug. 17, 2023

(54) TREATMENT OF INTERVERTEBRAL DISC DEGENERATION AND DISCOGENIC BACK PAIN

(71) Applicant: Kolon Tissuegene, Inc., Rockville, MD (US)

(72) Inventors: Moon Jong NOH, Rochville, MD (US); Huan T. Tran, Rockville, MD (US)

(73) Assignee: Kolon Tissuegene, Inc., Rockville, MD (US)

(21) Appl. No.: 18/134,401

(22) Filed: Apr. 13, 2023

Related U.S. Application Data
(63) Continuation-in-part of application No. 17/092,779, filed on Nov. 9, 2020.
Application No. 17/092,779 is a continuation of application No. PCT/US2020/025705, filed on Mar. 30, 2020.
(60) Provisional application No. 62/826,676, filed on Mar. 29, 2019.

(51) Int. Cl. A61K 38/18; A61K 35/32; A61K 35/22; A61K 47/20; A61K 47/26; A61P 19/02

(52) U.S. Cl. CPC A61K 38/1841; A61K 35/22; A61K 35/32; A61K 47/20; A61K 47/26; A61P 19/02

(57) ABSTRACT

A method for restoring a damaged or degenerating intervertebral disc, which includes administering a mixed cell composition containing mammalian connective tissue cells and mammalian cells expressing TGF-β1 into the intervertebral disc defect site.

### CROSS REFERENCE TO RELATED APPLICATIONS

This application is a continuation-in-part of U.S. Pat. Application No. 17/092,779 filed Nov. 9, 2020, which is a 371 National Stage Application of PCT/US2020/025705 filed Mar. 30, 2020, which claims priority from U.S. Provisional Application No. 62/826,676 filed Mar. 29, 2019.

### BACKGROUND

### Field

The present invention relates to prevention or retardation of intervertebral disc degeneration. The present application also relates to treating degenerating disc by preventing or retarding intervertebral disc degeneration. The present invention also relates to methods of using chondrocytes for introduction into injured intervertebral disc region and preventing or retarding degeneration of the intervertebral disc. The present invention also relates to a method of introducing at least one gene encoding a member of the transforming growth factor β superfamily into at least one mammalian cell for use in preventing or retarding degeneration of intervertebral disc in the mammalian host. The present invention also relates to a method of using a mixture of chondrocytes and mammalian cells expressing a gene encoding a member of the transforming growth factor β superfamily into injured intervertebral disc region and preventing or retarding degeneration of the intervertebral disc.

The present application also relates to prevention and treatment of back pain, such as low back pain associated with degeneration of intervertebral discs. The present application also relates to methods of using chondrocytes for introduction into injured intervertebral disc region and preventing or treating low back pain. The present invention also relates to a method of introducing at least one gene encoding a member of the transforming growth factor β superfamily into at least one mammalian cell for use in preventing or treating low back pain. The present invention also relates to a method of using a mixture of chondrocytes and mammalian cells expressing a gene encoding a member of the transforming growth factor β superfamily into intervertebral disc region and preventing or treating the low back pain, such as pain associated with degeneration of the intervertebral disc.

### SUMMARY

In one aspect, the present invention is directed to a method for preventing or retarding degeneration of intervertebral disc at an intervertebral disc defect site, which includes injecting a mammalian connective tissue cell into the intervertebral disc defect site. In another aspect, the present invention is directed to a method for preventing or treating chronic low back pain. In some aspects, the chronic low back pain is discogenic low back pain. In some aspects, the discogenic back pain is associated with or caused by degeneration of an intervertebral disc. The process preferably does not use a scaffolding or any supporting structure for the cells. That is, according to aspects of the present invention, the mammalian connective tissue cells are free of a scaffold or a three-dimensional support material that is pre-formed and shaped to accommodate the intended application, and the mammalian connective tissue cells do not contain deposits of cells formed by, for example, bioprinting. Preferably, non-transfected chondrocyte or fibroblast is used, and the subject is preferably a human being. If a chondrocyte is being used, the chondrocyte is preferably a non-disc chondrocyte or juvenile chondrocyte, meaning that the cells are isolated from a child who is less than two years old. In other aspects, the chondrocyte may be primed chondrocytes. In particular, the connective tissue cell may be allogeneic relative to the mammalian subject sought to be treated.

Transfected or transduced mammalian cells as discussed above may include epithelial cells, preferably human epithelial cells, or human embryonic kidney 293 cells, also referred to as HEK-293 or 293 cells.

In one aspect, the present invention relates to methods of using allogeneic juvenile chondrocytes or allogeneic non-disc chondrocytes for introduction into injured intervertebral disc region and preventing or retarding degeneration of the intervertebral disc.

In one aspect, the present invention relates to methods of using allogeneic juvenile chondrocytes or allogeneic non-disc chondrocytes for introduction into injured intervertebral disc region for preventing or treating the pain associated with degeneration of the intervertebral disc.

In one aspect, the present invention is used to prevent or retard further degeneration of an area in the intervertebral disc that has been injured, tom or herniated.

In one aspect, the present invention is used to prevent or treat the pain caused by degeneration of an area in the intervertebral disc that has been injured, tom or herniated.

In another aspect, the invention is directed to a method for preventing or retarding degeneration of intervertebral disc at an intervertebral disc defect site of a mammal, which method includes a) inserting a gene or a nucleic acid sequence encoding a protein having intervertebral disc regenerating function into a mammalian cell, and b) injecting the mammalian cell into the intervertebral disc defect site. The process preferably does not use a scaffolding or any supporting structure for the cells. In this method, the protein may belong to TGF-β superfamily, such as TGF-β, and preferably TGF-β1.

It is to be understood that whenever a reference is made to a gene encoding a protein having intervertebral disc regenerating function being transfected, transduced or inserted into a cell, another nucleic acid encoding said protein may be substituted for said gene. Such nucleic acid may be, for example, a coding region of a gene, an intron sequence, a recombinant sequence or a synthetic sequence.

In another aspect, the invention is directed to a method for preventing or treating the pain at an intervertebral disc defect site of a mammal, which method includes a) inserting a gene encoding a protein having intervertebral disc regenerating function into a mammalian cell, and b) transplanting the mammalian cell into the intervertebral disc defect site. The process preferably does not use a scaffolding or any supporting structure for the cells. The mammalian cells are free of a scaffold or a three-dimensional support material that is pre-formed and shaped to accommodate the intended application, and the mammalian cells do not contain deposits of cells formed by, for example, bioprinting. In this method, the protein may belong to TGF-β superfamily, such as TGF-β, and preferably TGF-β1.

Transfected mammalian cells as discussed above may include epithelial cells, preferably human epithelial cells, or human embryonic kidney 293 cells, also referred to as HEK-293 or 293 cells. In some aspects, the mammalian cell is GP2-293 packaging cells, also referred to as GP2-293 cells.

It is to be understood that when cells transfected with a gene or a nucleic acid sequence encoding a protein having intervertebral disc regenerating function are discussed herein, cells that are transduced with said gene or a nucleic acid sequence, or cells that have said gene or nucleic acid sequence inserted into their genome by other means or that express said exogenous gene or nucleic acid sequence may be substituted for said transfected cells.

In yet another aspect, the invention is directed to method for preventing or retarding degeneration of intervertebral disc at an intervertebral disc defect site of a mammal, which includes a) inserting a gene encoding a protein having intervertebral disc regenerating function into a first mammalian cell to give transfected mammalian cell, and b) transplanting a mixture of the transfected mammalian cell of a) and unmodified second mammalian cell that is a connective tissue cell into the intervertebral disc defect site. The process preferably does not use a scaffolding or any supporting structure for the cells. The first and the second mammalian cells are free of a scaffold or a three-dimensional support material that is pre-formed and shaped to accommodate the intended application, and the mammalian cells do not contain deposits of cells formed by, for example, bioprinting. In this method, the protein may belong to TGF-β superfamily, such as TGF-β, and preferably TGF-β1. In this method, the first mammalian cells may be epithelial cells, preferably human epithelial cells, or human embryonic kidney 293 cells, also referred to as HEK-293 or 293 cells. In some aspects, the mammalian cells are GP2-293 packaging cells, also referred to as GP2-293 cells.

In yet another aspect, the invention is directed to a method for preventing or treating pain at an intervertebral disc defect site of a mammal, which includes a) inserting a gene encoding a protein having an intervertebral disc regenerating function into a first mammalian cell to give transfected mammalian cell, and b) transplanting a mixture of the mammalian cell of a) and unmodified second mammalian cell that is a connective tissue cell into the intervertebral disc defect site. The process preferably does not use a scaffolding or any supporting structure for the cells. The first and the second mammalian cells are free of a scaffold or a three-dimensional support material that is pre-formed and shaped to accommodate the intended application, and the mammalian cells do not contain deposits of cells formed by, for example, bioprinting. In this method, the protein may belong to TGF-β superfamily, such as TGF-β, and preferably TGF-β1.

The first transfected mammalian cells as discussed above may include epithelial cells, preferably human epithelial cells, or human embryonic kidney 293 cells, also referred to as HEK-293 or 293 cells. In some aspects, the mammalian cells are GP2-293 packaging cells. The GP2-293 packaging cells are derived from HEK-293 cells which were modified to stably express retroviral gag and pol proteins. The GP2-293 packaging cells may be irradiated. The GP2-293 packaging cells are transduced to express a gene encoding a protein belonging to the TGF-β superfamily, such as TGF-β, and preferably TGF-β1.

In some aspects, the gene or a nucleic acid sequence encoding a protein belonging to the TGF-β superfamily may encode a mammalian protein, more specifically a human protein. In some aspects, the protein may be a recombinant protein.

The second mammalian cells may be connective tissue cells such as chondrocytes or fibroblasts. In the case of chondrocytes, the chondrocytes may be non-disc chondrocytes or juvenile chondrocytes. In particular, the chondrocytes for the second mammalian connective tissue cells may be primed chondrocytes. In another aspect, either or both of the first or second connective tissue cells may be allogeneic relative to the mammalian subject or to each other.

### BRIEF DESCRIPTION OF THE DRAWINGS

FIGS. 1A-1F show slowing, retardation, or prevention of degeneration of injured disc. (A) shows magnetic resonance imaging (MRI) radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Mixed cell treatment in particular, has an intervertebral anti-degenerating effect.

FIGS. 2A-2F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Mixed cell treatment in particular, has an intervertebral anti-degenerating effect.

FIGS. 3A-3D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. Mixed cell treatment in particular, has an intervertebral anti-degenerating effect.

FIGS. 4A-4D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. TGF-β1-producing 293 cells treatment in particular, has an intervertebral anti-degenerating effect.

FIGS. 5A-5D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. TGF-β1-producing 293 cells treatment and mixed cell treatments in particular, have an intervertebral anti-degenerating effect.

FIGS. 6A-6D show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. Untransduced chondrocytes treatment has an intervertebral anti-degenerating effect.

FIGS. 7A-7F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (C) shows MRI radiograph of a rabbit spine eight (8) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Untransduced chondrocytes treatment has an intervertebral anti-degenerating effect.

FIGS. 8A-8F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (C) shows MRI radiograph of a rabbit spine eight (8) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc. Untransduced chondrocytes treatment has an intervertebral anti-degenerating effect.

FIGS. 9A-9D show a slowing, retardation or prevention of degeneration of injured disc. (A) shows MRI radiograph of rabbit spine pre-surgery; (B) shows MRI radiograph of a rabbit spine eight (8) weeks after surgery in which (i) the disc at L2/3 was injured and cell culture media DMEM was injected, (ii) no puncture and no treatment control at spine locus L3/4, and (iii) disc at L4/5 was injured and primed chondrocytes were injected; arrows point to L2/3 and L4/5 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc. Primed chondrocyte treatment has an intervertebral anti-degenerating effect.

FIG. 10 shows experimental design of the study described in Example VI. In this and following figures, the term “TG-C” or “TGC” stands for the mixed cell composition used in the corresponding experiment.

FIGS. 11A-11C show the effect of the mixed-cell administration on the lumbar disc height as measured by the Disc Height Index (DHI) at 12 weeks (FIG. 11A), 24 weeks (FIG. 11B) or for both groups combined (FIG. 11C). The repeated ANOVA analysis of normalized %DHI indicated that the normalized %DHI in all TG-C treated groups is higher than in the CS10 group. The factorial analysis at the 12-week time point showed statistically higher values in the TG-C middle (P<0.01) and TG-C high (P<0.05) than in the CS10 group.

FIGS. 12A-12B show the result of the Pfirrmann grade of MRI image analysis used to evaluate disc degeneration. Twelve weeks following treatment, there was a significant difference between the CS10 and the TG-C mid group in the Pfirrmann grade (P<0.05, Kruskal Wallis test with the Bonferroni correction).

FIG. 13 shows mean T2 measurements of the discs at 12 weeks after injection of CS10 or mixed-cell low, mixed-cell mid, and mixed-cell high compositions. In FIG. 13, the term “TG-C” stands for the mixed cell composition. Annulus fibrosus is labeled as AF while nucleous pulposus is labeled as NP.

FIG. 14 shows mean T2 measurements of the discs at 24 weeks after injection of CS10 or mixed-cell low, mixed-cell mid, and mixed-cell high compositions. In FIG. 14, the term “TG-C” stands for the mixed cell composition. Annulus fibrosus is labeled as AF while nucleous pulposus is labeled as NP.

FIGS. 15A-15B show the result of the histological analyses of the L2/3 and L3/4 discs after the mixed-cell treatment. There were no statistically significant changes among the treatment groups at 12 weeks and 24 weeks after injection.

FIG. 16 shows disc height distribution (DHD) mapping from µCT for disc height analysis (panel a). Central 60 to 100% (panel b) and three topographic zones (panel c) were analyzed.

FIGS. 17A-17B show the result of the µCT scanning of discs. The data showed no significant difference among groups at each time point.

### DETAILED DESCRIPTION

As used herein, the term “biologically active” in reference to a nucleic acid, protein, protein fragment or derivative thereof is defined as an ability of the nucleic acid or amino acid sequence to mimic a known biological function elicited by the wild type form of the nucleic acid or protein.

As used herein, the term “mammalian cells” in reference to transfected or transduced cells includes all types of mammalian cells, in particular human cells, including but not limited to connective tissue cells such as fibroblasts or chondrocytes, or stem cells, and in particular human embryonic kidney cells, and further in particular, human embryonic kidney 293 cells, or epithelial cells.

As used herein, the term “connective tissue” is any tissue that connects and supports other tissues or organs, and includes but is not limited to a ligament, a cartilage, a tendon, a bone, and a synovium of a mammalian host.

As used herein, the term “connective tissue cell” or “cell of a connective tissue” include cells that are found in the connective tissue, such as fibroblasts, cartilage cells (chondrocytes), and bone cells (osteoblasts/osteocytes), which secrete collagenous extracellular matrix, as well as fat cells (adipocytes) and smooth muscle cells. Preferably, the connective tissue cells are fibroblasts, chondrocytes, or bone cells. More preferably, the connective tissue cells are chondrocytes cells. It will be recognized that the invention can be practiced with a mixed culture of connective tissue cells, as well as cells of a single type. Preferably, the connective tissue cell does not cause a negative immune response when injected into the host organism. It is understood that allogeneic cells may be used in this regard, as well as autologous cells for cell-mediated gene therapy or somatic cell therapy.

As used herein, “connective tissue cell line” includes a plurality of connective tissue cells originating from a common parent cell.

As used herein, “hyaline cartilage” refers to the connective tissue covering the joint surface. By way of example only, hyaline cartilage includes, but is not limited to, articular cartilage, costal cartilage, and nose cartilage.

In particular, hyaline cartilage is known to be self-renewing, responds to alterations, and provides stable movement with less friction. Hyaline cartilage found even within the same joint or among joints varies in thickness, cell density, matrix composition and mechanical properties, yet retains the same general structure and function. Some of the functions of hyaline cartilage include surprising stiffness to compression, resilience, and exceptional ability to distribute weight loads, ability to minimize peak stress on subchondral bone, and great durability.

Grossly and histologically, hyaline cartilage appears as a slick, firm surface that resists deformation. The extracellular matrix of the cartilage comprises chondrocytes, but lacks blood vessels, lymphatic vessels or nerves. An elaborate, highly ordered structure that maintains interaction between chondrocytes and the matrix serves to maintain the structure and function of the hyaline cartilage, while maintaining a low level of metabolic activity. The reference O′Driscoll, J. Bone Joint Surg., 80A: 1795-1812, 1998 describes the structure and function of hyaline cartilage in detail, which is incorporated herein by reference in its entirety.

As used herein, “injectable” composition refers to a composition that excludes various three-dimensional scaffold, framework, mesh or felt structure, which may be made of any material or shape that allows cells to attach to it and allows cells to grow in more than one layer, and which structure is generally implanted, and not injected. In one embodiment, the injection method of the invention is typically carried out by a syringe. However, any mode of injecting the composition of interest may be used. For instance, catheters, sprayers, or temperature dependent polymer gels also may be used.

As used herein, “juvenile chondrocyte” refers to chondrocyte obtained from a human being who is less than two years old. Typically, chondrocyte is obtained from preferably the hyaline cartilage region of an extremity of the body, such as a finger, nose, ear lobe and so forth. Juvenile chondrocytes may be used as donor chondrocytes for allogeneic treatment of defected or injured intervertebral disc.

As used herein, the term “mammalian host” includes members of the animal kingdom including but not limited to human beings.

As used herein, “mixed cell” or a “mixture of cells” or “cell mixture” refers to the combination of a plurality of cells that include a first population of cells that are transfected or transduced with a gene or a nucleic acid sequence of interest and a second population of cells that are not transduced or transfected.

In some embodiments of the invention, mixed cells may refer to the combination of a plurality of cells that include cells that have been transfected or transduced with a gene or a nucleic acid sequence encoding a member of the TGF-β superfamily and cells that have not been transfected or transduced with a gene or a nucleic acid sequence encoding a member of the TGF-β superfamily. Typically, the ratio of cells that have not been transfected or transduced with a gene or a nucleic acid sequence encoding a member of TGF-β superfamily to cells that have been transfected or transduced with a gene or a nucleic acid sequence encoding a member of the TGF-β superfamily may be in the range of about 1-20 to 1. The range may include about 3-10 to 1. In particular, the range may be about 3 to 1. However, it is understood that the ratio of these cells is not necessarily limited to any particular range so long as the combination of these cells is effective to treat an injured intervertebral disc by slowing or retarding its degeneration.

The effective dose or therapeutically effective dose of the mixed cells for mammals including human may be in a rage from about 0.1 × 10⁶ to about 100 × 10⁶ cells. In some embodiments, the effective dose or therapeutically effective dose of the mixed cells for mammals including human may be in a rage from about 0.5 × 10⁶ to about 50 × 10⁶ cells.

In embodiments, the cells including transfected and non-transfected cells, are free of a scaffold or a three-dimensional support material that is pre-formed and shaped to accommodate the intended application, and the cells do not contain deposits of cells formed by, for example, bioprinting.

As used herein, “non-disc chondrocyte” refers to chondrocytes isolated from any part of the body except for intervertebral disc cartilage tissue. Non-disc chondrocytes of the present invention may be used for allogeneic transplantation or injection into a patient to treat defected or injured intervertebral disc.

As used herein, the term “patient” includes members of the animal kingdom including but not limited to human beings.

As used herein, the term “primed” cell refers to cells that have been activated or changed to express certain genes.

As used herein, low back pain refers to as pain and discomfort, localized below the costal margin and above the inferior gluteal folds, with or without leg pain. Non-specific low back pain is defined as low back pain not attributed to recognizable, known specific pathology and specific low back pain which has known pathomorphological cause.

As used herein, discogenic back pain refers to back pain or discomfort associated with intervertebral disc degeneration without herniation, anatomical deformity, or other alternate clear causes of pain and disability.

As used herein, discogenic low back pain refers to discogenic back pain localized below the costal margin and above the inferior gluteal folds, with or without leg pain.

As used herein, “slowing” or “prevention” of intervertebral disc degeneration refers to the retention of volume of intervertebral disc or height of the disc over time compared with the volume or height level that would normally be found at the site of injury leading to normal degeneration over a given time. This may mean a percentage increase of volume or height, such as about 10%, 20%, 30%, 40%, 50%, 60%, 70%, 80%, or 90% compared with the normal expected degeneration levels at a given time or may mean lessening of damage or depletion of volume or height of the intervertebral disc at the locus.

As used herein, the “transforming growth factor-β (TGF-β) superfamily” encompasses a group of structurally related proteins, which affect a wide range of differentiation processes during embryonic development. The family includes, Müllerian inhibiting substance (MIS), which is required for normal male sex development (Behringer, et al., Nature, 345:167, 1990), Drosophila decapentaplegic (DPP) gene product, which is required for dorsal-ventral axis formation and morphogenesis of the imaginal discs (Padgett, et al., Nature, 325:81-84, 1987), the Xenopus Vg-1 gene product, which localizes to the vegetal pole of eggs (Weeks, et al., Cell, 51:861-867, 1987), the activins (Mason, et al., Biochem, Biophys. Res. Commun., 135:957-964, 1986), which can induce the formation of mesoderm and anterior structures in Xenopus embryos (Thomsen, et al., Cell, 63:485, 1990), and the bone morphogenetic proteins (BMP’s, such as BMP-2, 3, 4, 5, 6 and 7, osteogenin, OP-1) which can induce de novo cartilage and bone formation (Sampath, et al., J. Biol. Chem., 265:13198, 1990). The TGF-β gene products can influence a variety of differentiation processes, including adipogenesis, myogenesis, chondrogenesis, hematopoiesis, and epithelial cell differentiation (for a review, see Massague, Cell 49:437, 1987), which is incorporated herein by reference in its entirety.

The proteins of the TGF-β family are initially synthesized as a large precursor protein, which subsequently undergoes proteolytic cleavage at a cluster of basic residues approximately 110-140 amino acids from the C-terminus. The C-terminal regions of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. In each case, the active species appears to be a disulfide-linked dimer of C-terminal fragments. For most of the family members that have been studied, the homodimeric species has been found to be biologically active, but for other family members, like the inhibins (Ung, et al., Nature, 321:779, 1986) and the TGF-β′s (Cheifetz, et al., Cell, 48:409, 1987), heterodimers have also been detected, and these appear to have different biological properties than the respective homodimers.

Members of the superfamily of TGF-β genes or proteins include TGF-β3, TGF-β2, TGF-β4 (chicken), TGF-β1, TGF-β5 (Xenopus), BMP-2, BMP-4, Drosophila DPP, BMP-5, BMP-6, Vgrl, OP-1/BMP-7, Drosophila 60A, GDF-1, Xenopus Vgf, BMP-3, Inhibin-βA, Inhibin-βB, Inhibin-α, and MIS. These genes and proteins are discussed in Massague, Ann. Rev. Biochem. 67:753-791, 1998, which is incorporated herein by reference in its entirety.

Preferably, the member of the superfamily of TGF-β genes and proteins is TGF-β1, TGF-β2, TGF-β3, BMP-2, BMP-3, BMP-4, BMP-5, BMP-6, or BMP-7.

### Intervertebral Disc

The intervertebral discs make up one fourth of the spinal column’s length. There are no discs between the Atlas (C1), Axis (C2), and Coccyx. Discs are not vascular and therefore depend on the end plates to diffuse needed nutrients. The cartilaginous layers of the end plates anchor the discs in place.

The intervertebral discs are fibrocartilaginous cushions serving as the spine’s shock absorbing system, which protect the vertebrae, brain, and other structures (i.e. nerves). The discs allow some vertebral motion: extension and flexion. Individual disc movement is very limited - however considerable motion is possible when several discs combine forces.

Intervertebral discs are composed of an annulus fibrosus and a nucleus pulposus. The annulus fibrosus is a strong radial tire-like structure made up of lamellae; concentric sheets of collagen fibers connected to the vertebral end plates. The sheets are orientated at various angles. The annulus fibrosus encloses the nucleus pulposus.

Although both the annulus fibrosus and nucleus pulposus are composed of water, collagen, and proteoglycans (PGs), the amount of fluid (water and PGs) is greatest in the nucleus pulposus. PG molecules are important because they attract and retain water. The nucleus pulposus contains a hydrated gel-like matter that resists compression. The amount of water in the nucleus varies throughout the day depending on activity. As people age, the nucleus pulposus begins to dehydrate, which limits its ability to absorb shock. The annulus fibrosus gets weaker with age and begins to tear. While this may not cause pain in some people, in others one or both of these may cause chronic back pain.

Pain due to the inability of the dehydrating nucleus pulposus to absorb shock is called axial pain or disc space pain. One generally refers to the gradual dehydration of the nucleus pulposus as degenerative disc disease. When the annulus fibrosus tears due to an injury or the aging process, the nucleus pulposus can begin to extrude through the tear. This is called disc herniation. Near the posterior side of each disc, all along the spine, major spinal nerves extend out to different organs, tissues, extremities etc. It is very common for the herniated disc to press against these nerves (pinched nerve) causing radiating pain, numbness, tingling, and diminished strength and/or range of motion. In addition, the contact of the inner nuclear gel, which contains inflammatory proteins, with a nerve can also cause significant pain. Nerve-related pain is called radicular pain.

Intervertebral disc may be damaged by an injury or trauma or degenerated by aging. Damaged or degenerating intervertebral disc may be, but is not limited to, a thinning disc or a herniated disc. According to the embodiments of the present disclosure, the damaged or degenerating intervertebral disc may be restored structurally by increasing the height (or height index) and/or the volume of the damaged or degenerating disc (by, for example, increasing the content of water, collagen matrix, and/or proteoglycan in the damaged or degenerating disc) by administering an effective amount of a mixed cell composition as described herein to the damaged or degenerating intervertebral disc site. The administration route may be, but is not limited to, topical injection or transplantation of the mixed cell composition to the target damaged or degenerating disc site.

Herniated discs may be referred to by many names and these can mean different things to different medical professionals. A slipped disc, ruptured disc, or a bulging disc can all refer to the same medical condition. Protrusions of the disc into the adjacent vertebra are known as Schmorl’s nodes.

### Primed Cell Therapy

The present invention encompasses administering primed cells to an intervertebral disc region in a mammal to treat injured intervertebral disc by preventing or retarding degeneration of intervertebral disc. Primed cells are typically connective tissue cells, and include chondrocytes or fibroblasts.

The present invention encompasses administering primed cells to an intervertebral disc region in a mammal to restore the structure of the damaged or degenerating intervertebral disc. According to an embodiment, the damaged or degenerating intervertebral disc could be thinning disc or herniated disc. Primed cells are typically connective tissue cells, and include chondrocytes or fibroblasts.

By way of example, when a population of primary chondrocytes are passaged about 3 or 4 times, their morphology typically changes to fibroblastic chondrocytes. As primary chondrocytes are passaged, they begin to lose some of their chondrocytic characteristics and begin to take on the characteristics of fibroblastic chondrocytes. When these fibroblastic chondrocytes are incubated or “primed” with a cytokine such as a protein from the TGF-β superfamily, the cells regain their chondrocytic characteristics, which include production of extracellular matrix such as collagens and proteoglycans.

Such primed cells include fibroblastic chondrocytes, which have been incubated with TGF-β1, and as a result have reverted to collagen producing chondrocytes. An advantage of using primed cells in retardation of intervertebral disc degeneration is the ease of creating useable chondrocytes for introduction into the intervertebral disc for production of collagen and otherwise maintenance of the cartilaginous matrix, and effectively relieving back pain.

The cells may include without limitation primary cells or cells which have undergone about one to twenty passages. The cells may be connective tissue cells. The cells may include cells that have undergone a morphogenic change, wherein the priming causes reversion to the characteristics of the original cell. The cells may include without limitation chondrocytes, fibroblasts, or fibroblastic chondrocytes. Priming may occur by incubating the cells for a period of at least 40 hours, or from 1 to 40 hours, from 2 to 30 hours, from 3 to 25 hours, from 4 to 20 hours, from 5 to 20, from 6 to 18 hours, 7 to 17 hours, 8 to 15 hours, or 9 to 14 hours, with a cytokine, and then optionally separating the cytokine from the cells and injecting the primed cells into a cartilaginous defect site of interest in order to regenerate cartilage, preferably hyaline cartilage. In one aspect, the cytokine may be a member of the superfamily of TGF-β. In particular, the cytokine may be TGF-β, and in particular, TGF-β1.

The cytokine may be present in the priming incubation mix in an amount to sufficiently “prime” the chondrocyte to be useful in the intervertebral treatment method. In this aspect, the priming incubation mix may contain at least about 1 ng/ml of the cytokine. In particular, the mix may contain from about 1 to 1000 ng/ml, from about 1 to 750 ng/ml, from about 1 to 500 ng/ml, from about 1 to 400 ng/ml, from about 1 to 300 ng/ml, from about 1 to 250 ng/ml, from about 1 to 200 ng/ml, from about 1 to 150 ng/ml, from about 1 to 100 ng/ml, from about 1 to 75 ng/ml, from about 1 to 50 ng/ml, from about 10 to 500 ng/ml, from about 10 to 400 ng/ml, from about 10 to 300 ng/ml, from about 10 to 250 ng/ml, from about 10 to 200 ng/ml, from about 10 to 150 ng/ml, from about 10 to 100 ng/ml, from about 10 to 75 ng/ml, from about 10 to 50 ng/ml, from about 15 to 500 ng/ml, from about 15 to 400 ng/ml, from about 15 to 300 ng/ml, from about 15 to 250 ng/ml, from about 15 to 200 ng/ml, from about 15 to 150 ng/ml, from about 15 to 100 ng/ml, from about 15 to 75 ng/ml, from about 15 to 50 ng/ml, from about 20 to 500 ng/ml, from about 20 to 400 ng/ml, from about 20 to 300 ng/ml, from about 20 to 250 ng/ml, from about 20 to 200 ng/ml, from about 20 to 150 ng/ml, from about 20 to 100 ng/ml, from about 20 to 75 ng/ml, from about 20 to 50 ng/ml, from about 25 to 500 ng/ml, from about 25 to 400 ng/ml, from about 25 to 300 ng/ml, from about 25 to 250 ng/ml, from about 25 to 200 ng/ml, from about 25 to 150 ng/ml, from about 25 to 100 ng/ml, from about 25 to 75 ng/ml, from about 25 to 50 ng/ml, from about 30 to 500 ng/ml, from about 30 to 400 ng/ml, from about 30 to 300 ng/ml, from about 30 to 250 ng/ml, from about 30 to 200 ng/ml, from about 30 to 150 ng/ml, from about 30 to 100 ng/ml, from about 30 to 75 ng/ml, from about 30 to 50 ng/ml, from about 35 to 500 ng/ml, from about 35 to 400 ng/ml, from about 35 to 300 ng/ml, from about 35 to 250 ng/ml, from about 35 to 200 ng/ml, from about 35 to 150 ng/ml, from about 35 to 100 ng/ml, from about 35 to 75 ng/ml, from about 35 to 50 ng/ml, from about 40 to 500 ng/ml, from about 40 to 400 ng/ml, from about 40 to 300 ng/ml, from about 40 to 250 ng/ml, from about 40 to 200 ng/ml, from about 40 to 150 ng/ml, from about 40 to 100 ng/ml, from about 40 to 75 ng/ml, or from about 40 to 50 ng/ml.

One method of practicing the invention may include incubating the cells with a cytokine for a certain length of time to create primed cells and optionally separating the cytokine from the cells, and injecting the primed cells into intervertebral disc or the site of interest near it. Alternatively, the cells may be incubated with the cytokine of interest for a time and the combination may be administered to the site of defect without separating out the cytokine.

It is to be understood that while it is possible that substances such as a scaffolding or a framework as well as various extraneous tissues may be implanted together in the primed cell therapy protocol of the present invention, it is also possible that such scaffolding or tissue not be included in the injection system of the invention. In a preferred embodiment, in the inventive somatic cell therapy, the invention is directed to a simple method of injecting a population of primed connective tissue cells to the intervertebral disc space.

It will be understood by the artisan of ordinary skill that the source of cells for treating a human patient may be the patient’s own cells, but that allogeneic cells as well as xenogeneic cells may also be used without regard to the histocompatibility of the cells. Alternatively, in one embodiment of the invention, allogeneic cells may be used having matching histocompatibility to the mammalian host. To describe in further detail, the histocompatibility of the donor and the patient are determined so that histocompatible cells are administered to the mammalian host. Also, juvenile chondrocytes may also be used allogeneically without necessarily determining the histocompatibility of the donor and the patient.

### Gene Delivery

In one aspect the present invention discloses ex vivo and in vivo techniques for delivery of a DNA sequence of interest to the connective tissue cells of the mammalian host. The ex vivo technique involves culture of target mammalian cells, in vitro transfection of the DNA sequence, DNA vector or other delivery vehicle of interest into the mammalian cells, followed by transplantation of the modified mammalian cells to the target area of the mammalian host, so as to effect in vivo expression of the gene product of interest.

It is to be understood that while it is possible that substances such as a scaffolding or a framework as well as various extraneous tissues may be implanted together in the protocol of the present invention, it is preferred that such scaffolding or tissue not be included in the injection system of the invention. In a one embodiment, the invention is directed to a simple method of injecting a TGF superfamily protein or a population of cultured, untransfected/untransduced connective tissue cells or transfected/transduced mammalian cells or a mixture thereof to the intervertebral disc space so that the exogenous TGF superfamily protein is expressed or is active in the space.

It will be understood by the artisan of ordinary skill that one source of cells for treating a human patient is the patient’s own cells. Another source of cells includes allogeneic cells without regard to the histocompatibility of the cells to the patient sought to be treated.

More specifically, this method includes employing a gene product that is a member of the transforming growth factor β superfamily, or a biologically active derivative or fragment thereof, or a biologically active derivative or fragment thereof.

In another embodiment of this invention, a composition for parenteral administration to a patient in a therapeutically effective amount is provided that contains a TGF-β superfamily protein and a suitable pharmaceutical carrier.

Another embodiment of this invention provides a composition for parenteral administration to a patient in a prophylactically effective amount that includes a TGF-β superfamily protein and a suitable pharmaceutical carrier.

In therapeutic applications, the TGF-β protein may be formulated for localized administration. Techniques and formulations generally may be found in Remington’s Pharmaceutical Sciences, Mack Publishing Co., Easton, Pa., latest edition. The active ingredient that is the TGF protein is generally combined with a carrier such as a diluent or excipient which may include fillers, extenders, binding, wetting agents, disintegrants, surface-active agents, erodable polymers, lubricants, preservatives, such as cryopreservatives, and others. A suitable carrier may be selected depending on the mode of administration and dosage form. The carrier may be or may contain a cryopreservation medium. The cryopreservation medium may contain about 10 to 20% w/w dimethyl sulfoxide (DMSO) and about 1 to 5 w/w% saccharose. Typical dosage forms include, powders, liquid preparations including suspensions, emulsions, solutions, granules, and capsules.

The TGF protein of the present invention may also be combined with a pharmaceutically acceptable carrier for administration to a subject. Examples of suitable pharmaceutical carriers are a variety of cationic lipids, including, but not limited to N-(1-2,3-dioleyloxy)propyl)-n,n,n-trimethylammonium chloride (DOTMA) and dioleoylphophotidyl ethanolamine (DOPE). Liposomes are also suitable carriers for the TGF protein molecules of the invention. Another suitable carrier is a slow-release gel or polymer comprising the TGF protein molecules.

The TGF beta protein may be mixed with an amount of a physiologically acceptable carrier or diluent, such as a saline solution or other suitable liquid. The TGF protein molecule may also be combined with other carrier means to protect the TGF protein and biologically active forms thereof from degradation until they reach their targets and/or facilitate movement of the TGF protein or biologically active form thereof across tissue barriers.

A further embodiment of this invention includes storing the cell prior to transferring the cells. It will be appreciated by those skilled in the art that the cells may be stored frozen in 10 percent DMSO in liquid nitrogen.

In the present application, a method is provided for regenerating or preventing degeneration of intervertebral disc, or for preventing or treating back pain, by injecting an appropriate mammalian cell that is transfected or transduced with a gene or a nucleic acid sequence encoding a member of the transforming growth factor-beta (TGF-β) superfamily, including, but not limited to, BMP-2 and TGF-β 1, 2, and 3.

In another embodiment of the present application, a method is provided for preventing or retarding degeneration of intervertebral disc, or for preventing or treating back pain, by injecting an appropriate connective tissue cell that is not transfected or transduced with a gene or a nucleic acid sequence encoding a member of the transforming growth factor-beta (TGF-β) superfamily or that is not transfected or transduced with any other gene. In another aspect, the invention is directed to treating injured or degenerated intervertebral disc by preventing or retarding degeneration of the intervertebral disc by using the above-described method.

In another embodiment of the present application, a method is provided for preventing or retarding degeneration of intervertebral disc, or for preventing or treating back pain, by injecting a combination of or a mixture of an appropriate mammalian cell that is transfected or transduced with a gene or a nucleic acid sequence encoding a member of the transforming growth factor-beta (TGF-β) superfamily and an appropriate connective tissue cell that is not transfected or transduced with a gene or a nucleic acid sequence encoding a member of the transforming growth factor-beta (TGF-β) superfamily or that is not transfected or transduced with any other gene. In another aspect, the invention is directed to treating injured or degenerated intervertebral disc by preventing or retarding degeneration of the intervertebral disc by using the above-described method.

In an embodiment of the invention, it is understood that the cells may be injected into the area in which degeneration of the intervertebral disc is to be sought to be prevented or retarded by using the cell above-described composition with or without scaffolding material or any other auxiliary material, such as extraneous cells or other biocompatible carriers. That is, the cells are free of a scaffold or a three-dimensional support material that is pre-formed and shaped to accommodate the intended application, and the mammalian cells do not contain deposits of cells formed by, for example, bioprinting. Thus, the modified cells alone, unmodified cells alone, or a mixture or combination thereof may be injected into the area in which the degeneration of the intervertebral disc is sought to be prevented or retarded, or the site of pain where the pain to be mitigated or alleviated.

The following examples are offered by way of illustration of the present invention, and not by way of limitation.

### EXAMPLES

### Example I - Materials and Methods

### Plasmid Construction

The plasmid pMTMLVβ1 was generated by subcloning a 1.2-kb Bgl II fragment containing the TGF-β1 coding sequence and a growth hormone poly A site at the 3′ end into the Bam HI site of pMTMLV. pMTMLV vector was derived from the retroviral vector MFG by deleting entire gag and env sequences as well as some of ψ packaging sequence.

### Cell Culture and Transduction

The TGF-β cDNA cloned in retroviral vectors were individually transduced into 293 cells (293-TGF-β1). They were cultured in Dulbecco’s Modified Eagle’s Medium (GIBCO-BRL, Rockville, MD) with 10% concentration of fetal bovine serum.

To select the cells transduced with the TGF-β1 coding sequence, neomycin (300 µg/ml) was added into the medium. The cells with TGF-β1 expression were sometimes stored in liquid nitrogen and cultured just before the injection.

### Radiographic Analysis of Disc Height

Radiographs were taken after administration of ketamine hydrochloride (25 mg/kg) and Rompun (1 mg/kg) at various week intervals after the puncture. Extreme care was taken to maintain a consistent level of anesthesia during radiography of each animal and at each time to obtain a similar degree of muscle relaxation, which may affect the disc height. Therefore, the preoperative radiograph was always used as a baseline measurement. Efforts were also made to keep the spine in a slightly flexed position. To decrease the error from axial rotation of the spine and beam divergence, radiographs were repeated at least twice on each animal in the lateral decubitus position, with the beam centered at 4 cm from the rabbit iliac crest. Radiographs were digitally scanned and digitally stored using an Image Capture software.

### Image Analysis

Using digitized radiographs, measurements, including the vertebral body height and intervertebral disc (IVD) height, were analyzed using the public domain image analysis. The data were transported to Excel software, and the IVD height was expressed as the disc height index (DHI) using the method of Lu et al. “Effects of chondroitinase ABC and chymopapain on spinal motion segment biomechanics. An in vivo biomechanical, radiologic, and histologic canine study”, Spine 1997;22:1828-34. Average IVD height (DHI) was calculated by averaging the measurements obtained from the anterior, middle, and posterior portions of the IVD and dividing that by the average of adjacent vertebral body heights. Changes in the DHI of injected discs were expressed as percent DHI and normalized to the measured preoperative IVD height (percent DHI = postoperative DHI/preoperative DHI X 100). The within-subject standard deviation (Sw) was calculated using the equation:

$$√ \left(\sum {\left({\text{x}}_{1} - {\text{x}}_{2}\right)}^{2} / 2 \text{n}\right)$$

Where X₁ is the first measurement value, X₂ is the second measurement value, and n = 450. The percent coefficient of variance (percent CV) was calculated as (Sw/means of all measurements X 100). The intraobserver error of DHI measurements was estimated to be minimal (Sw: 0.001800316; percent CV: 3.13). The interobserver error was also reported to be small (Sw: 0.003227; percent CV: 9.6)

### MRI Assessments

MRI examinations were performed on all rabbits in the study using a 0.3-T imager (Airis II, version 4.0 A; Hitachi Medical System America, Inc.) with a quadrature extremity coil receiver. After sacrifice, the spinal columns with surrounding soft tissue were isolated and subjected to MRI analysis. T2-weighted sections in the sagittal plane were obtained in the following settings: fast spin echo sequence with TR (time to repetition) of 4000 milliseconds and TE (time to echo) of 120 milliseconds; 256(h) X 128 (v) matrix; field of view of 260; and 4 excitations. The section thickness was 2 mm with a 0-mm gap. A blinded observer using the modified Thompson classification based on changes in the degree and area of signal intensity from grade 1 to 4 (1 = normal, 2 = minimal decrease of signal intensity but obvious narrowing of high signal area, 3 = moderate decrease of signal intensity, and 4 = severe decrease of signal intensity) evaluated MRIs. The intraobserver and interobserver reliability correlation coefficients of MRI grading based on 2 evaluations were excellent (K = 0.98, 0.90, respectively), as determined by the Cohen kappa correlation coefficient.

### Example II - Experimental Methods and Results

### Preventing Degeneration of Injured Intervertebral Disc

New Zealand white male rabbits were used. An open surgical technique was used. Three intervertebral levels in the lumbar spine: L2-3, L3-4, L4-5 were experimentally treated or observed as a control in each animal. Treatments were assigned to levels in a balanced manner with multiple sites/discs per rabbit observed. Within subject design, pre- post-surgery comparisons, change across disc levels were used as controls.

### Example III

### Preventing Degeneration of Injured Intervertebral Disc Using Untransduced Chondrocyte Alone, TGF-Bl-Producing 293 Cells Alone, or With Mixed-Cells (Human Chondrocytes and TGF-B1-Producing 293 Cells) Injection in Rabbits

All of the chondrocytes used in Examples I-V are non-disc chondrocytes and are juvenile chondrocytes, obtained from the hyaline cartilage portion of a finger of a less than two year old child.

Needle puncture was produced in the intervertebral discs of the lumbar spine. After this needle puncture, TGF-β1-producing 293 cells, primary untransduced human chondrocytes, a mixture of TGF-β1-producing 293 cells and primary untransduced human chondrocytes, primed untransduced human chondrocytes or carrier/media are injected. Several controls are used. Experimental conditions are listed in Table I.

TABLE 1

| | |
|---|---|
| Surgical Preparation | Injection Treatment |
| Needle puncture | TGF-β1-producing 293 cells (~5 × 10⁶cells) |
| Needle Puncture | Mixed: TGF-β1-producing 293 cells Primary untransduced human chondrocytes (~3 to 1 ratio, 5 × 10⁶) |
| Needle Puncture | Primary untransduced human chondrocytes (~5 × 10⁶) |
| Needle Puncture | Primed untransduced human chondrocytes (~5 × 10⁶) |
| Needle puncture | DMEM |
| Needle puncture | Needle puncture only-no injection |
| No puncture | No puncture no treatment control |

Briefly, a needle puncture injury is produced in the intervertebral discs of the lumbar spine of rabbit or a pig. After this needle puncture, rabbits are left to heal for 4 weeks. Then in a second surgical procedure, experimental treatment composition, which includes TGF-β1-producing 293 cells and/or primary untransduced human chondrocytes (~5 × 10⁵) is injected or control conditions observed (Table I).

After endotrachial intubation and general anesthesia is achieved such as by administration of ketamine hydrochloride and ROMPUN®, the animal is placed in supine position. Lactated ringers are used at about (5 ml/kg/hr). The area of incision is shaved and prepped and draped in the usual sterile fashion with alternating betadine scrubs and alcohol wipes (> three times). Bland ophthalmic ointment is placed on the eyes. A left retroperitoneal approach is used to expose the right anterior aspect of the disc from L2-L5 (the rabbit has 6 to 7 lumbar vertebra). Various preparation schemes are used and treatment schema is applied to each disc level. For ‘Needle Puncture’ preparation of the disc, a 18-gauge needle is used to place a puncture in the disc at the depth of 5 mm (Aoki et al., “Nerve fiber ingrowth into scar tissue formed following nucleus pulposus extrusion in the rabbit anular-puncture disc degeneration model: effects of depth of puncture.” Spine. 2006;31(21):E774-80). After puncture, the test materials listed in Table I are injected. Treatment composition is applied to any one of L1-2, L2-3, L3-4, L4-5 region of each rabbit.

Monthly radiographs are used to monitor any disc changes. Animals are sacrificed at 2, 8, and 24 weeks after surgery.

Radiographs/MRI. Healing is indicated by a detectable radiographic change of increased disc height from same disc at baseline (pre op) compared to disc at other disc levels. Other discs are compared before and after needle puncture only, and disc before and after no needle puncture yielding an index of normal degeneration over time.

Retro-Transcription PCR. Retro-transcription PCR is performed to assay relative quantity of surviving transfected chondrocytes.

Histology. Also histology is used to confirm characterization of the collagen type I and type II and the gross appearance and evaluation of de novo chondrocytes.

Western Blot analysis and or ELISA. Quantatitive expression of collagen type I and type II, and proteoglycan concentration, Smads ⅔, Sox-9. Additionally ELISA is used to evaluate TGFβ-1, BMP2, BMP7, GDF5 and other related growth factors where there are available antibodies.

Apoptosis is examined in the other tissue structures of the intervertebral disc via observing the expression of Capase-3.

### Example IV

### Results

The results are as shown in the Figures and the description of the Figures of the present application. Punctured intervertebral disc treated with untransduced chondrocytes alone, transduced 293 cells alone, primed chondrocyte alone or a mixture of transduced 293 cells and untransduced chondrocytes, show beneficial effects in preventing or retarding disc degeneration compared with vehicle control.

### Example IV-1 - Mixed-Cell (Transduced 293 Cells and Untransduced Chondrocytes) Treatment of Punctured Intervertebral Disc in Rabbit

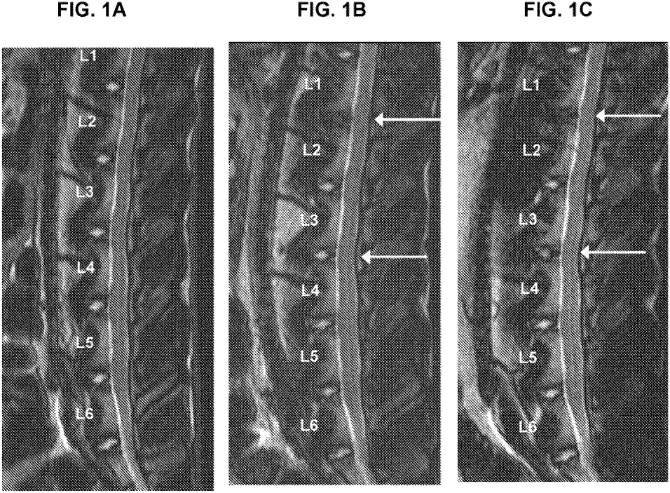

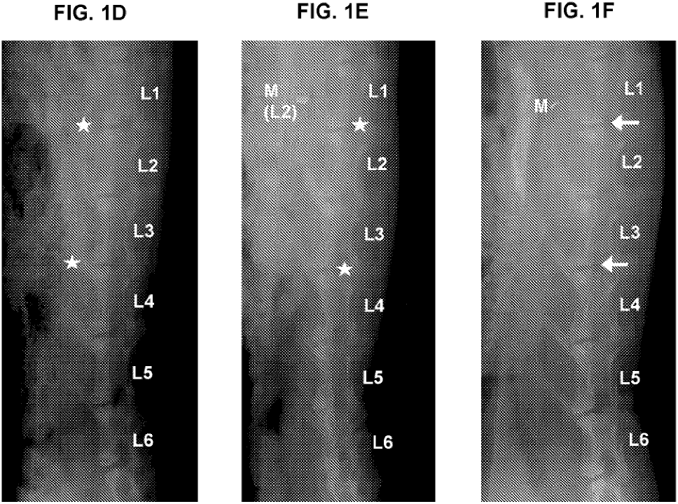

Mixed cell treatment has an intervertebral anti-degenerating effects when tested on rabbits. The effect is seen in a variety of experiments in FIGS. 1-4. FIGS. 1A-1F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

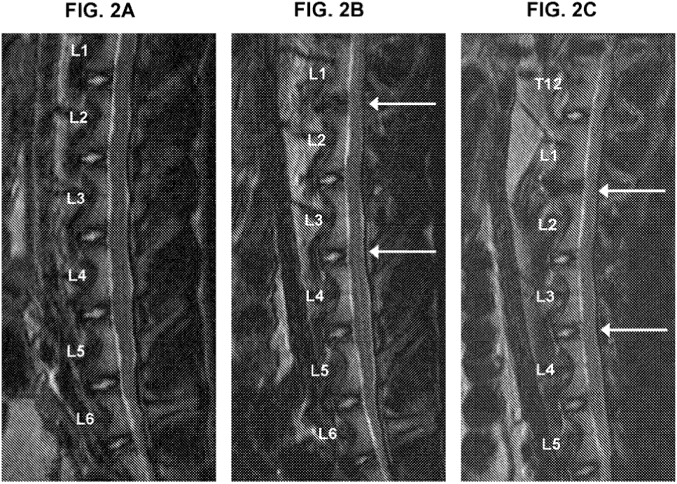

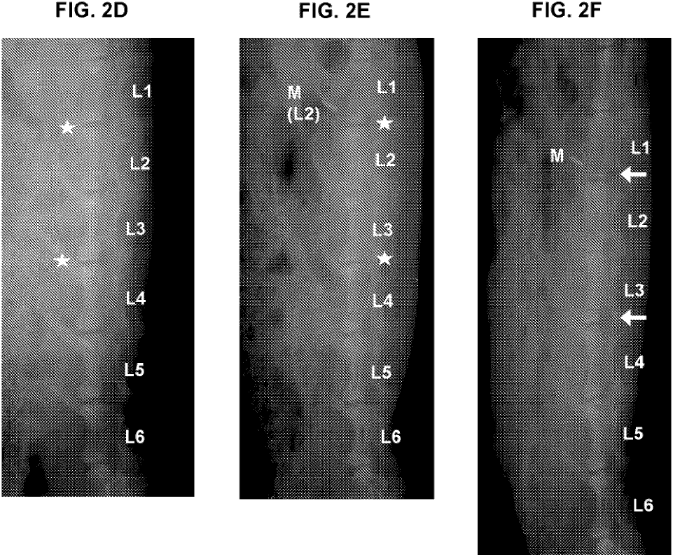

FIGS. 2A-2F show slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows MRI radiograph of a rabbit spine eight (8) weeks after surgery in which (i) the disc at L1/2 was injured and TGF-β1-producing 293 cells were injected, (ii) no puncture and no treatment is seen at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

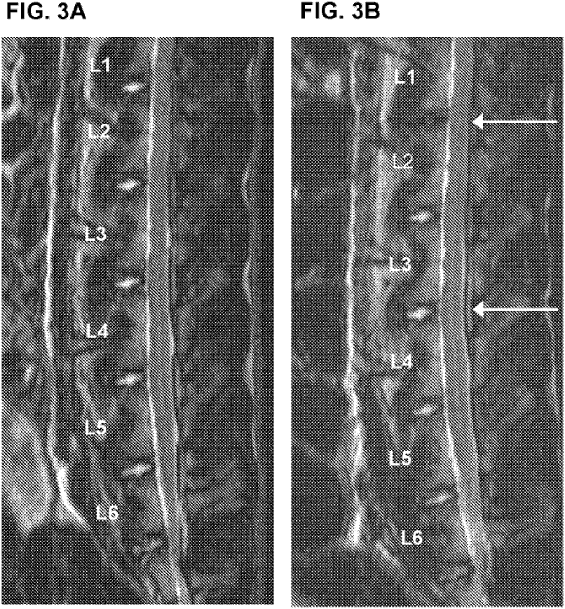

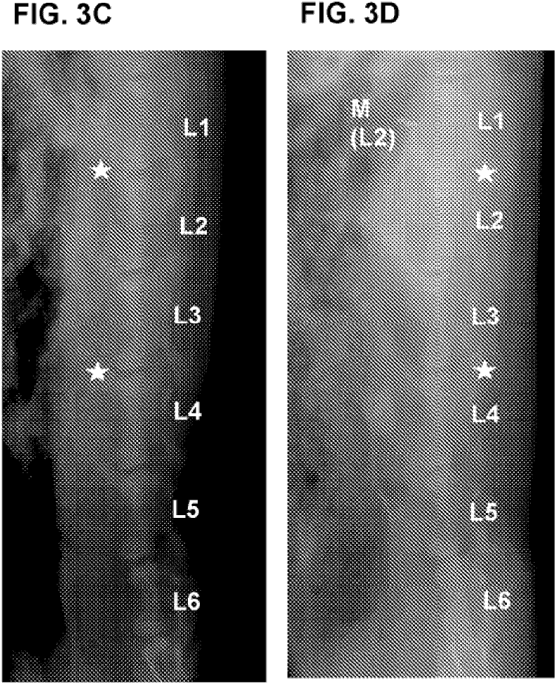

FIGS. 3A-3D show slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and TGF-β1-producing 293 cells were injected, (ii) no puncture and no treatment control at spine locus L2/3, and (iii) disc at L3/4 was injured and mixture of TGF-β1-producing 293 cells and untransduced human chondrocytes in 1:3 ratio were injected; arrows point to L1/2 and L3/4 disc region. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example IV-2 - Transduced 293 Cell Treatment of Punctured Intervertebral Disc in Rabbit

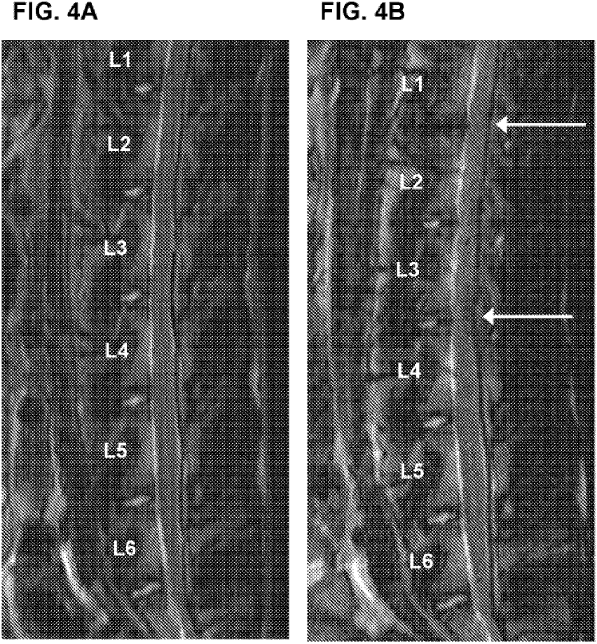

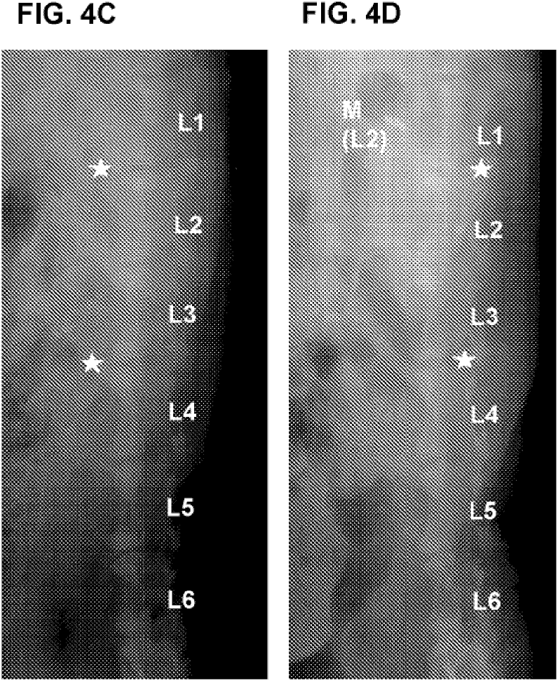

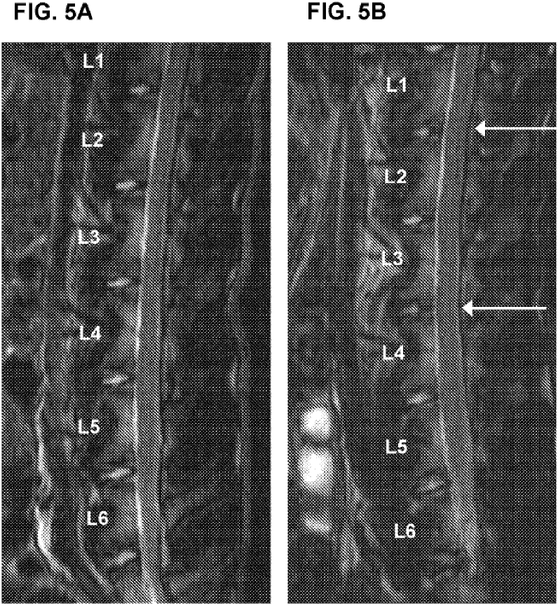

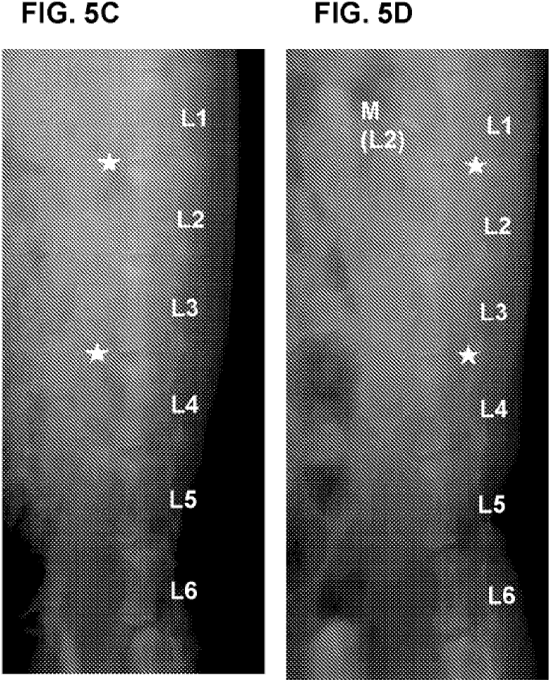

TGF-β1-producing 293 cells treatment has an intervertebral anti-degenerating effect. The effect is seen in FIGS. 4A-4D, which show slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example IV-3 - Transduced 293 Cell Treatment and Mixed-Cell Treatment of Punctured Intervertebral Disc in Rabbit

TGF-β1-producing 293 cell treatment and mixed cell treatments have an intervertebral anti-degenerating effect. The effect is seen in FIGS. 5A-5D, which show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and mixture of TGF-β1-producing 293 cells and untransduced human chondrocytes in 1:3 ratio were injected, (ii) no puncture and no treatment control at spine locus L2/3, and (iii) disc at L3/4 was injured and TGF-β1-producing 293 cells were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example IV-4 - Untransduced Chondrocyte Treatment of Punctured Intervertebral Disc in Rabbit

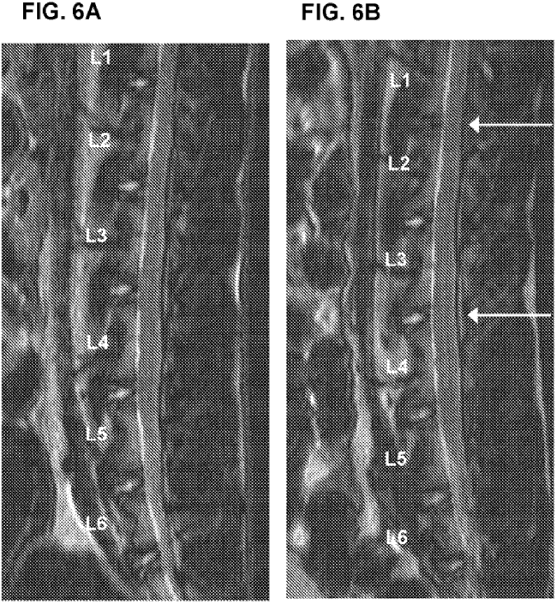

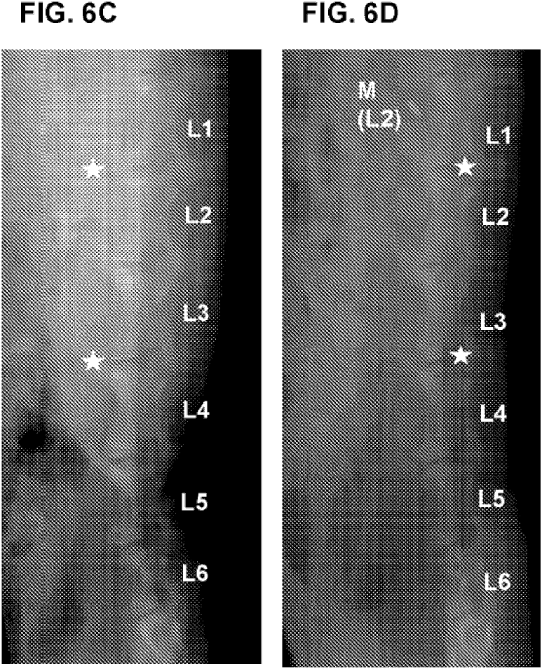

Untransduced chondrocyte treatment has an intervertebral anti-degenerating effect. The effect is seen in a variety of experiments in FIGS. 6-8. FIGS. 6A-6D show slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2, was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

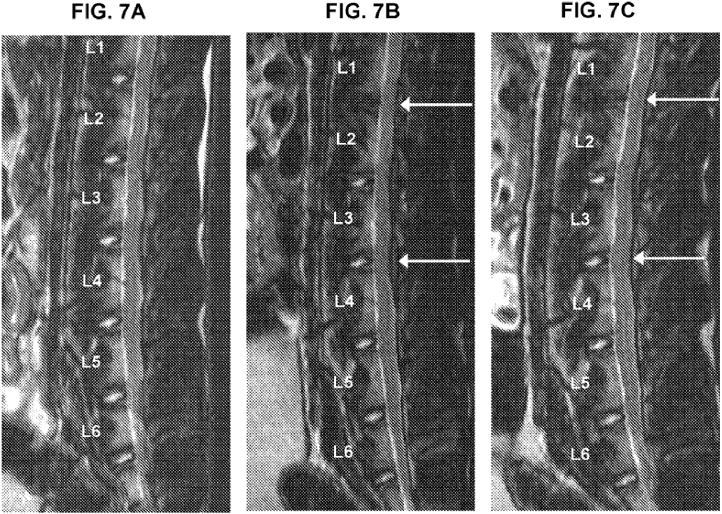

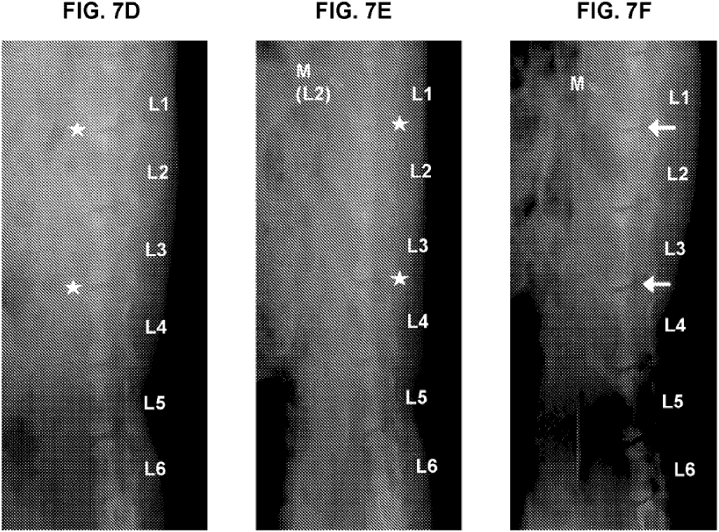

FIGS. 7A-7F show a slowing, retardation or prevention of degeneration of injured disc. (A) shows MRI radiograph of rabbit spine pre-surgery; (B) shows MRI radiograph of a rabbit spine four (4) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (C) shows MRI radiograph of a rabbit spine eight (8) weeks after surgery in which (i) the disc at L1/2 was injured and cell culture media DMEM was injected, (ii) no puncture and no treatment control at spine locus L2/3, and (iii) disc at L3/4 was injured and untransduced chondrocytes were injected; arrows point to L1/2 and L3/4 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

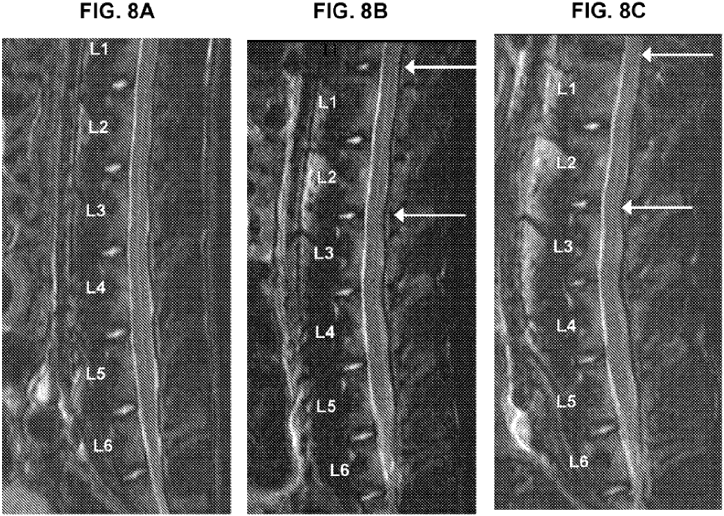

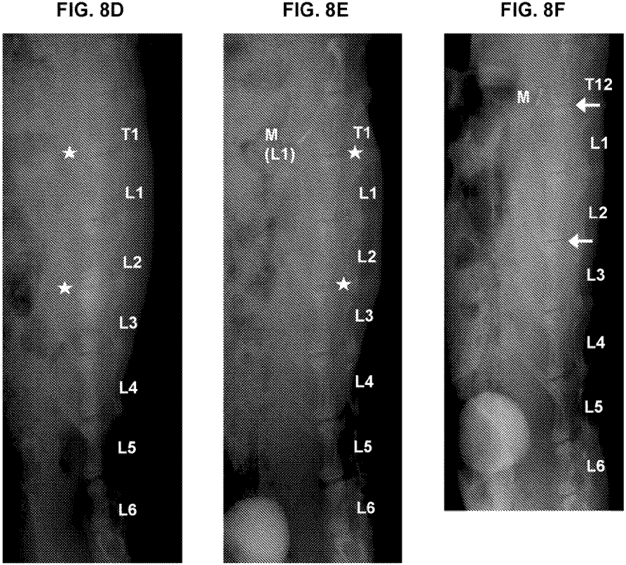

FIGS. 8A-8F show slowing, retardation or prevention of degeneration of injured disc. (A) shows MRI radiograph of rabbit spine pre-surgery; (B) shows MRI radiograph of a rabbit spine four (4) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (C) shows MRI radiograph of a rabbit spine eight (8) weeks after surgery in which (i) the disc at T12/L1 was injured by needle puncture and no injection, (ii) no puncture and no treatment control at spine locus L1/2, and (iii) disc at L2/3 was injured and untransduced chondrocytes were injected; arrows point to T12/L1 and L2/3 disc regions. (D) shows X-ray radiograph of the rabbit described in (A) above, which is used to obtain a disc height index of the intervertebral disc to measure its morphology, its level of degeneration or regeneration. (E) shows X-ray radiograph of the rabbit described in (B) above, which is used to obtain a disc height index of the intervertebral disc. (F) shows X-ray radiograph of the rabbit described in (C) above, which is used to obtain a disc height index of the intervertebral disc.

### Example IV-5 - Untransduced Primed Chondrocyte Treatment of Punctured Intervertebral Disc in Rabbit

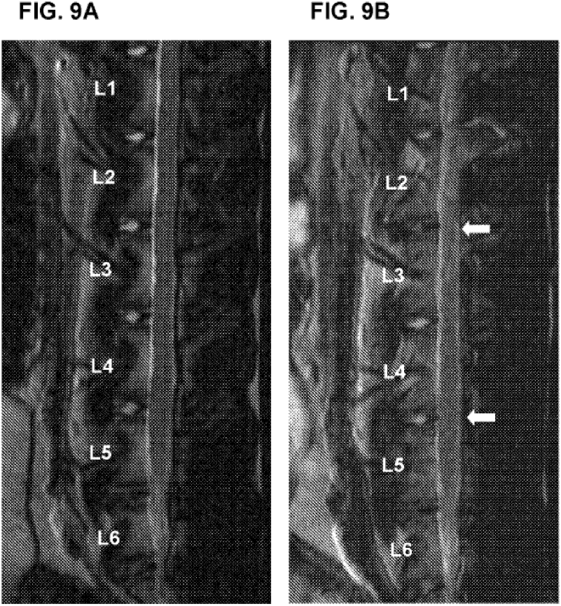

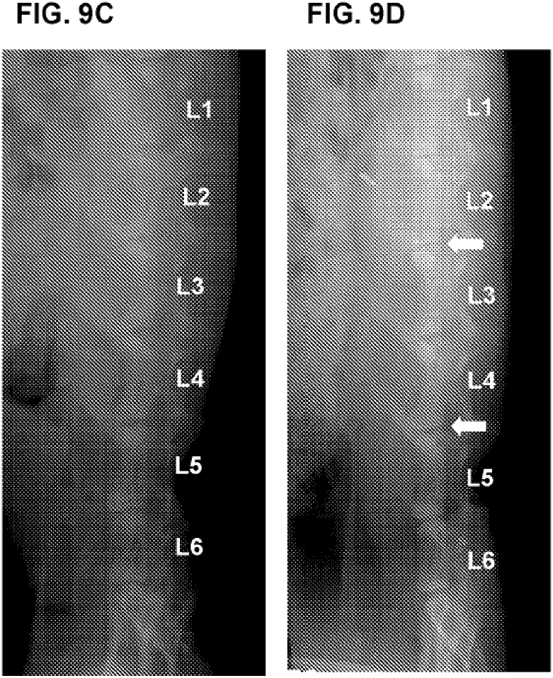

Primed chondrocyte treatment has an intervertebral anti-degenerating effect. The effect is seen in FIGS. 9A-9D, which show slowing, retardation or prevention of degeneration of injured disc. (A) shows MRI radiograph of rabbit spine pre-surgery; (B) shows MRI radiograph of a rabbit spine eight (8) weeks after surgery in which (i) the disc at L2/3 was injured and cell culture media DMEM was injected, (ii) no puncture and no treatment control at spine locus L3/4, and (iii) disc at L4/5 was injured and primed chondrocytes were injected; arrows point to L2/3 and L4/5 disc regions. (C) shows X-ray radiograph of the rabbit described in (A) above, which is used to obtain a disc height index of the intervertebral disc to measure its morphology, its level of degeneration or regeneration. (D) shows X-ray radiograph of the rabbit described in (B) above, which is used to obtain a disc height index of the intervertebral disc.

### Example V

### Source of Human Chondrocytes

Primary human chondrocytes were grown from cartilage tissue obtained from the surgical excision of a polydactyly finger from a one-year-old female human donor. The polydactyl tissue was harvested in a surgical room. The following procedure for chondrocyte isolation was performed in a biosafety cabinet. The plastic bottle containing the cartilage tissue was swiped with alcohol and the cartilage tissue was washed with sterile PBS (1X) using a pipette. A collagenase solution was prepared by dissolving 7 mg of collagenase (Gibco BRL) in 10 mL of DMEM (containing 10% FBS) and filtering through a 0.2 µm syringe filter (Coming). The washed cartilage tissue was treated with the collagenase solution for 17 to 18 hrs in a 37° C. shaker incubator. On the following day, the bottle was sanitized with alcohol. The collagenase treated material was pipetted up and down several times to separate loose cells from the tissue mass. After pipetting, the supernatant was filtered through 70 µm nylon cell strainer (Falcon). Collagenase treated tissue which had lost its integrity (e.g., loose cells) was able to pass through the filter. The cell filtrate was collected in a 50 mL tube (Falcon) and then centrifuged at 1,500 rpm for 5 minutes. Two thirds of the supernatant was discarded and the pellet washed with 10 ml of sterile PBS (1X). The resuspended cells were again centrifuged at 1,500 rpm for 5 minutes and, after removal of two-thirds of the supernatant, washed with 10 ml of sterile PBS (1X). The cells were again centrifuged at 1,500 rpm for 5 minutes and then resuspended in DMEM (containing 10% FBS). The resuspended cells were then transferred to four uncoated 25 cm² flasks and cultured for four days at 37° C. with 5% CO₂. The cells were then transferred into two uncoated 185 cm² flasks. The cells were cultured for two weeks and then collected, washed and resuspended in a cryopreservative media of DMEM, FBS and DMSO in a 5:4:1 ratio. The cells were aliquotted in to cryovials containing 1 mL of cell suspension at 4 × 10⁵ cells/mL. The cells were held in vapor phase liquid nitrogen storage.

### Example VI - Intradiscal Injection of Mixed-Cell Compositions in Rabbit Anular Puncture Model

### Experimental Design

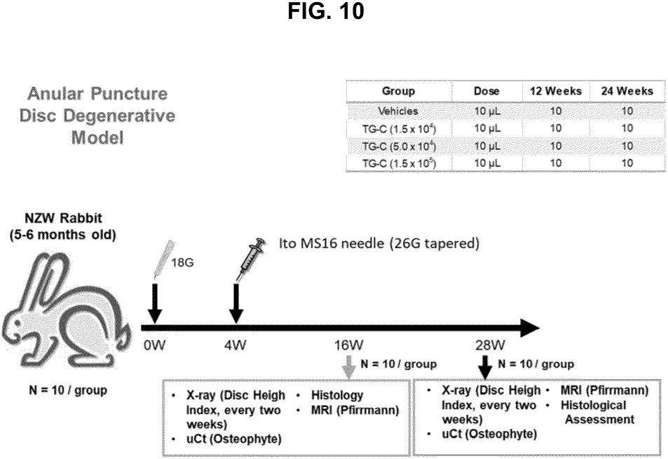

Anular puncture induces a prolapsed nucleus pulposus (NP) at the punctured site and depressurization of the NP, resulting in degenerative changes in both annulus fibrosus (AF) and NP. This study was conducted to investigate whether an intradiscal injection of mixed cells inhibits or reverses the disc degeneration in rabbit anular puncture model. The design of the study is shown in FIG. 10.

Eighty New Zealand White rabbits weighing approximately 3.3-4.5 kg (5-6 months old) were used. At the time of the initial puncture, two non-contiguous discs were punctured by an 18 Gauge needle, with the disc between the punctured discs left intact as a control. Four weeks after the initial puncture, the animals were divided into four groups of 20 and each of the two punctured discs was injected with either CS10 (10 µl), or mixed cells at the following doses: 1.5 × 10⁴ (mixed-cell low composition or TG-C low), 5 × 10⁴ (mixed-cell mid composition or TG-C mid), and 1.5 × 10⁵ (mixed-cell high composition or TG-C high). The extent of degeneration was monitored radiographically throughout the study. After 12 and 24 weeks, rabbits (10 per group) were sacrificed subjected to MRI and micro-computerized tomography (µCT) analysis (Yamaguchi et al., Trans Orthop Res Soc, 2011, 2011:173). The punctured/treated discs (L2/3, L4/5) and control discs (L3/4) were collected for histological analyses. The Experimental groups are shown in Table II.

TABLE II

| | | | |
|---|---|---|---|
| Group | Dose | 12 weeks | 24 weeks |
| CS10 | V=10 µl | 10 female rabbits per group | 10 female rabbits per group |
| TG-C low | 1.5 × 10⁴ per disc in V=10 µl | | |
| TG-C mid | 5 × 10⁴ per disc in V=10 µl | | |
| TG-C high | 1.5 × 10⁵ per disc in V=10 µl | | |

A comparison among the groups was made using ANOVA for parametric analysis and Mann-Whitney test for non-parametric analysis.

### Surgical Procedure for the Rabbit IVD Degeneration Model

Female New Zealand White rabbits (specific pathogen-free (SPF), 5-6 months-old animals) were used. One to five days before the surgery, a pre-operative X-ray was taken as a baseline control under anesthesia by administering ketamine hydrochloride (25 mg/kg) and acepromazine maleate (1 mg/kg, 10 mg/ml) or isoflurane anesthesia. Animals received a fentanyl patch (12.5 µg/h) one day before surgery, given preoperatively as preemptive analgesia. A dose of ketamine hydrochloride (35 mg/kg) and xylazine (5 mg/kg) was given intramuscularly. Animals were intubated and maintained by isoflurane inhalation (induced at 2-3% and maintained at 2-5%). The rabbits received 50 ml of fluids (Lactated Ringer’s solution or NaCl) subcutaneously while being prepped for surgery.

The rabbits were placed in a lateral prone position. Following prepping and draping, the lumbar IVDs were exposed through a posterolateral retroperitoneal approach by blunt dissection of the psoas muscle. The anterior surfaces of three consecutive lumbar IVDs (L2/3, L3/4, and L4/5) were exposed. Using an 18G needle with a stopper device that allows the needle to penetrate to a depth of 5 mm, the AF was punctured in the ventral aspect into the NP at the L2/3 and L4/5 levels. A titanium staple and a black silk suture (2-0) were placed at the L3/4 level as a reference point. The surgical wound created was then repaired in layers and the skin was closed using staples. During or after the surgery, an intra/post- operative X-ray was taken to confirm the level of puncture.

An application of a fentanyl patch was used to allow the animal to wake up from surgery with little or no discomfort. The fentanyl patch was left on the rabbits for three days post-operatively to help minimize pain and distress. The animals were given a dose of cefazolin (22 mg/kg, SQ) before the surgery for prophylaxis purposes. After recovery from anesthesia, the rabbits were returned to their cages and mobilized ad-lib.

### Surgical Procedure for Mixed-Cell Injection

Four weeks after the initial surgery (anular puncture), a similar surgical procedure was performed from the opposite side to avoid bleeding from the scar formed from the first operation. Once the surgically degenerating discs were confirmed by X-ray and visual inspection, test materials were intradiscally injected into the NP area with a microsyringe (MS*GFN25, Ito Corporation, Fuji, Japan) using a XX*MS16 needle (Ito Corporation) at both the L2/3 and L4/5 levels for each rabbit. A previous study has shown minimal damage to IVDs injected with a XX*MS11 (OD 0.52 mm, close to 25G) needle. XX*MS16 has a tapered needle tip, which is equivalent to 26G needle. After repair of the surgical wound, the rabbits were returned to their cages and closely monitored. The behavior, appetite, and change in body weight were closely monitored and the level appropriateness of surgical stress was evaluated by the animal care veterinary staff and the investigators.

### Mixed-Cell Treatment

Mixed-cell treatments contained 1.5 × 10⁴ total cells per disc (mixed-cell low, also referred to as TG-C Low), 5.0 × 10⁴ total cells per disc (mixed-cell mid, also referred to as TG-C Mid), or 4.5 × 10⁵ total cells per disc (mixed-cell high, also referred to as TG-C High) containing a mixture of human allogeneic chondrocytes and irradiated GP2-293 cells expressing TGF-β1 at the ratio of 3 to 1. CRYOSTOR® CS10, BioLifeSolution, WA, USA, was used as a vehicle control.

### Radiographic Analysis

Every two weeks after the initial anular puncture, an X-ray to measure IVD height was taken. Typically, X-rays were taken after administering ketamine hydrochloride (25 mg/kg) and acepromazine maleate (1 mg/kg) or under isoflurane anesthesia. Extreme care was taken to maintain a consistent level of anesthesia during the radiography of each animal and at each time point in order to obtain a similar degree of muscle relaxation, which may affect the disc height. The pre-operative X-ray was always used as a baseline measurement. Various efforts were made to keep the spine in a straight position. In order to reduce the error from the axial rotation of the spine and beam divergence, radiographs were repeated at least twice on each animal in the lateral decubitus position with the beam centered at 4 cm from the rabbit iliac crest.

### X-Ray Image Analysis

All X-ray images were independently interpreted by an orthopedic researcher who was blinded to the study group, surgical procedure, and time point. Using digitized X-rays, measurements, including the vertebral body height and IVD height, were analyzed using the custom program for MATLAB software (Natick, MA). Data was exported to Excel software, and the IVD height expressed as the disc height index (DHI = IVD height / adjacent IVD body height) based on the previously developed method (Masuda et al., Spine 30(1): 5-14, 2005). The mean IVD height (DHI) was calculated by averaging the measurements obtained from the anterior, middle, and posterior portions of the IVD and dividing that by the mean of the adjacent vertebral body heights. Changes in the DHI of injected discs was expressed as %DHI which was calculated by dividing the post-operative DHI with pre-operative DHI.

$$\text{\%DHI} = \frac{P o s t - o p e r a t i v e D H I}{P r e - o p e r a t i v e D H I} x 100$$

The normalized %DHI was determined using the non-punctured L3/4 level as control as shown in the following formulae (Mwale et al. Arthritis Research & Therapy, 13(4): R120, 2011):

$$\text{Normalized} \text{\%DHI} = \frac{P u n c t u r e d \% D H I}{N o n - p u n c t u r e d \% D H I} x 100.$$

### Euthanasia and Sample Preparation

At 16 or 32 weeks after the initial anular puncture for those rabbits receiving treatments (12 or 24 weeks after injections) 10 rabbits in each group were anesthetized with ketamine hydrochloride (25 mg/kg) and acepromazine maleate (1 mg/kg) and euthanatized with an excess dose of pentobarbital (Euthanasia B solution: Henry Schein Inc., Melville, NY).

### MRI Assessment

### Image Acquisition

MRI examinations on dissected specimens were performed on all rabbits in the study using a BIOSPEC 70/30 USR (Bruker, ON). After sacrifice, the spinal columns, with surrounding soft tissue were isolated and imaged. Two types of pulse sequences were used to obtain T2-weighted images for traditional IVD grading and T2 quantification for quantification of MRI parameters.

For anatomic images and Pfirrmann grading, T2-weighted images in the sagittal plane were obtained using the following settings: fast spin-echo sequence with TR (time to repetition) = 3000 ms, TE (time to echo) = 100 ms, matrix = 512 × 256, FOV=10, NEX=8, and slice thickness = 1 mm. Six slices were obtained with a 0 mm gap. Scanning parameters are shown in Table III.

For T2 quantification, a constant TR and multiple TE sequence were used to obtain 16 images at different TE. The following setting was used: TR=1000 ms, TE (16 echoes) = 13, 25, 38, 51, 64, 76, 89, 102, 114, 127, 140, 152, 165, 178, 191, 203.

TABLE III

| | | | | |
|---|---|---|---|---|
| Mid-sagittal Scan | 2 mm slice thickness, 5 slices | FOV 6.93 × 4.30 cm | 273 × 230 matrix | 186 × 187 µm in plane resolution |
| Axial Scan | 2 mm slice thickness, 3 slices | FOV 5.60 × 3.45 cm | 37 3× 230 matrix | 150 × 150 µm in plane resolution |

Note: the detailed condition may be altered to obtain the highest quality image.

### MRI Grade - Pfirrmann Classification

For semi-quantitative morphologic assessment, T2-weighted images were evaluated by three observers blinded to the experiment using the Pfirrmann classification based on changes in the degree and area of signal intensity from grades 1-5 (Pfirrmann et al., Spine 26(17): 1873-78, 2001). Pfirrmann Classification of Disc Degeneration is shown in Table IV. The grades from the three observers were averaged to represent the grade for each disc.

TABLE IV

Classification of Disc Degeneration ^{∗}

| | | | | |
|---|---|---|---|---|
| Grade | Structure | Distinction of Nucleus and Anulus | Signal Intensity | Height of Intervertebral Disc |
| I | Homogeneous, bright while | Clear | Hyperintense, isointense to cerebrospinal fluid | Normal |
| II | Inhemogeneous with or without horizontal bands | Clear | Hyperintense, isointense to cerebrospinal fluid | Normal |
| III | Inhomogeneous, gray | Unclear | Intermediate | Normal to slightly decreased |
| IV | Inhomogeneous, gray to black | Lost | Intermediate to hypointense | Normal to moderately decreased |
| V | Inhomogeneous, black | Lost | Hypointense | Collapsed disc space |

[symbol]

[symbol]

indicates text missing or illegible when filed

### Image Processing & Analyses

For T2 quantification, the MR images at multiple TE were imported into MATLAB with Image Processing Toolbox, and region representing the NP was segmented to place region of interest for T2 calculation and to measure the area of the NP. The intensity values within ROIs were averaged for each image and then fit to Equation [1] for each ROI. In addition, T2 maps were determined by fitting each voxel using Equation [1] as well.

$$\text{S} \left(T E\right) = {\text{S}}_{0} exp \left(\text{-} T E / T 2\right) \qquad \text{[1]}$$

For T1rho quantification, a similar approach to T2 quantification was used, fitting ROI and voxel intensity to Equation [2] to obtain T1rho values and maps, respectively.

$$\text{S} \left(S T L\right) = {\text{S}}_{0} exp \left(\text{-} S T L / T 1 r h o\right) \qquad \text{[2]}$$

### Histological Analyses of the Intervertebral Disc

The IVDs or samples from in vitro experiments were fixed in 10% neutralized formalin, decalcified with Cal-Ex™ II Fixative/Decalcifier and embedded in paraffin. Sagittal sections (5-8 µm) of each IVD were stained with hematoxylin and eosin (cellular constituents), and Safranin-O (proteoglycans). To grade disc degeneration, a separate observer, blinded to the treatment group, graded the discs using an established protocol. Chujo et al., Spine, 2006, 31(25): 2909-17; Gullbrand et al., JOR spine, 2021, 4(2): e114).

### Results

### Surgery Schedule and Complications

The surgery was performed in two days for each time point. All animals maintained their health status during the study except for some wounds and appetite loss after surgeries. These are typical outcomes of multiple surgeries. There were no statistically significant differences among the treatment group at puncture, an injection, and sacrifice as shown in Tables V and VI.

TABLE V

Body Weight of animals (the 12-week group)

| Group | Puncture BW (±SE) | Injection BW | Sacrifice BW |
|---|---|---|---|
| CS10 | 4.41 ± 0.11 | 4.08 ± 0.09 | 4.48 ± 0.10 |
| TGC Low | 4.16 ± 0.09 | 3.87 ± 0.04 | 4.27 ± 0.06 |
| TGC Mid | 4.24 ± 0.11 | 3.99 ± 0.12 | 4.45 ± 0.08 |
| TGC High | 4.26 ± 0.14 | 3.98 ± 0.11 | 4.41 ± 0.12 |

TABLE VI

Body Weight of animals (the 24-week group)

| Group | Puncture BW | Injection BW | Sacrifice BW |
|---|---|---|---|
| CS10 | 4.22 ± 0.08 | 4.22 ± 0.09 | 4.61 ± 0.11 |
| TGC Low | 4.37 ± 0.14 | 4.25 ± 0.07 | 4.74 ± 0.1 |
| TGC Mid | 4.16 ± 0.1 | 4.13 ± 0.1 | 4.65 ± 0.1 |
| TGC High | 4.11 ± 0.06 | 4.06 ± 0.08 | 4.61 ± 0.05 |

### Exclusions From the Final Analyses

In the 12-week group, the Disc Height Index at the 4-week time point indicated that the puncture of some discs in the CS10 (two discs), TG-C low (two discs), and TG-C mid (one disc) did not effectively cause degeneration (criteria: normalized %DHI less than 95% at four weeks after puncture). These discs were eliminated from further evaluation as non-responders. One animal might have received the puncture at the wrong levels, and these discs were removed from the analysis.

In the 24-week group, some discs in the TG-C low (two discs), TG-C mid (three discs), TG-C high (three discs) have been excluded for the same reason.

### Radiographic Analysis of Disc Height Index

### 12-Week Group

The initial anular injury was induced in rabbit discs of 40 NZW rabbits; this procedure resulted in degenerative changes in the nucleus pulposus (NP) and anulus fibrosus (AF). After four weeks, the rabbits were injected with vehicle control (CS10) or TG-C cells (low, mid and high in 10 µl CS10) and followed up by X-ray biweekly. Twelve weeks after cells or vehicle injection, animals were sacrificed, followed by magnetic resonance imaging (MRI), and the discs were subjected to histological analysis.

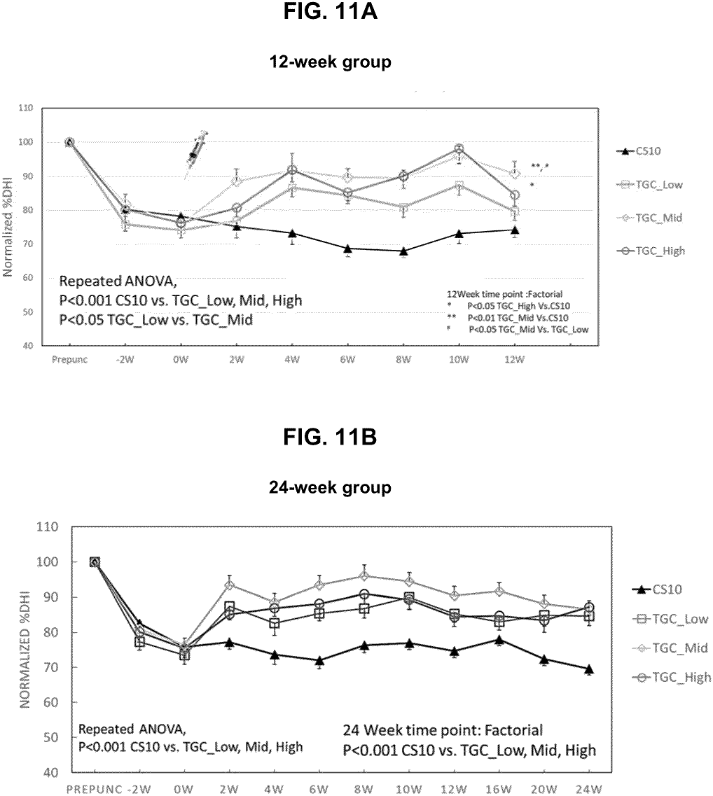

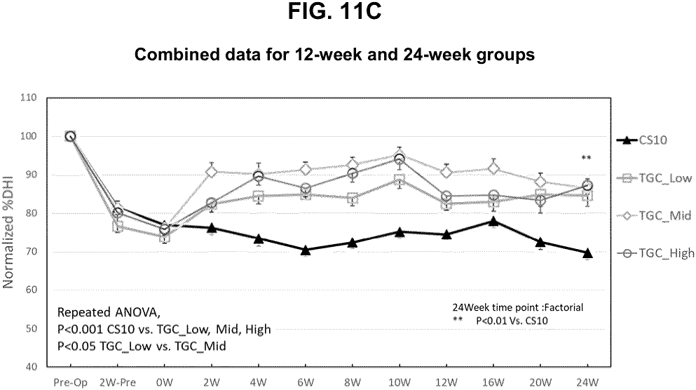

The repeated ANOVA analysis of normalized %DHI indicated that the normalized %DHI in all TG-C injected groups is higher than in the CS10 group. The factorial analysis at the 12-week time point showed statistically higher values in the TG-C middle (P<0.01) and TG-C high (P<0.05) than in the CS10 group. The results are shown in FIG. 11A.

### 24-Week Group

Another set of rabbits (40 NZW rabbits) underwent the same needle puncture procedure and injection with vehicle control (CS10), or TG-C cells (low, mid and high in 10 µl CS10) four weeks after the initial puncture. Twenty-four weeks later, the animals were sacrificed, followed by magnetic resonance imaging (MRI), and the discs were subjected to histological analysis. The repeated ANOVA analysis of normalized %DHI of all TG-C injected groups after two weeks was significantly higher than that of the CS10 group (p<0.001). The statistical result at the 24-week time point showed all groups with TG-C injections were higher than the vehicle control group (CS10). The results are shown in FIG. 11B.

### 12- and 24-week Groups Combined Data

The combined data of the 12- and 24-week groups showed essentially the same results as shown in the 24-month group. The DHI results are shown in FIG. 11C.

### Radiographic Analysis of Disc Degeneration Using Pfirrmann Disc Grading System

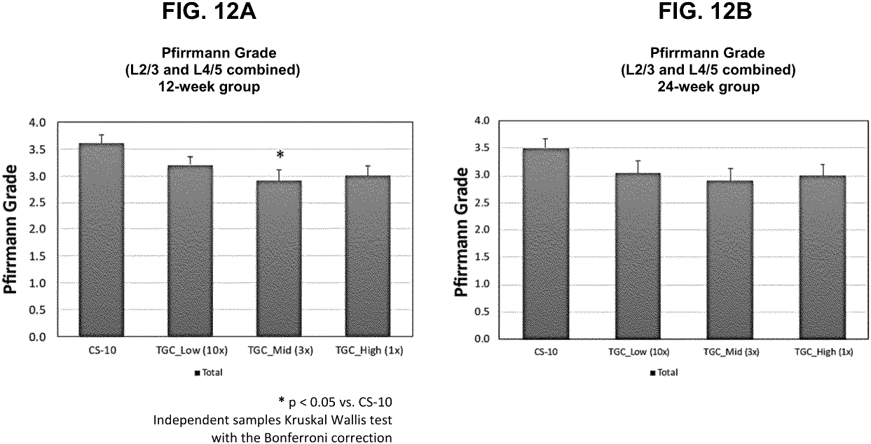

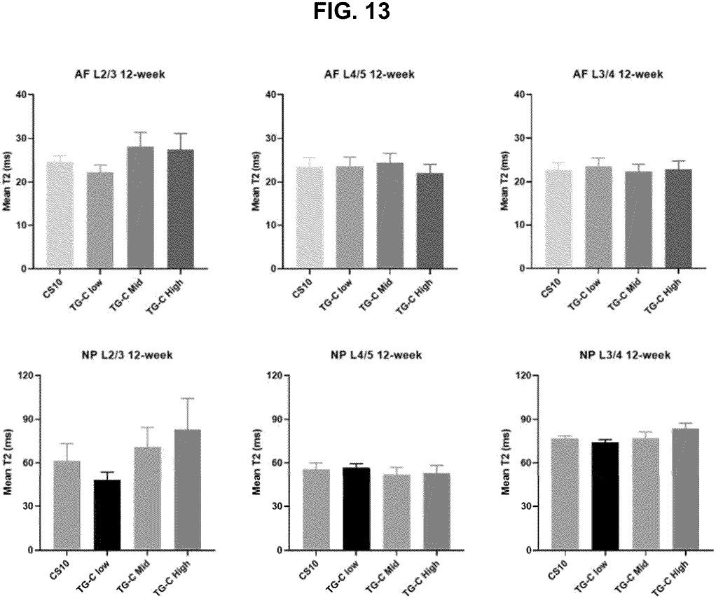

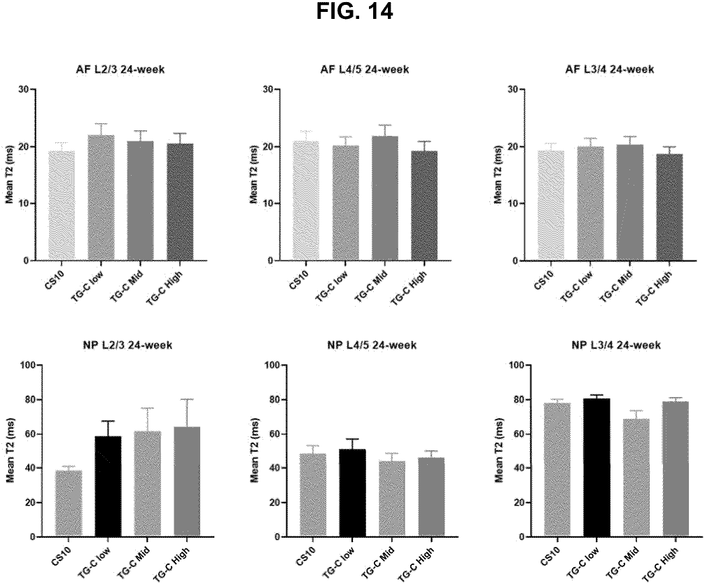

Pfirrmann grade analysis was used to evaluate disc degeneration. Twelve weeks following treatment, there was a significant difference between the CS10 and the TG-C mid group in the Pfirrman grade (P < 0.05, Kruskal Wallis test with the Bonferroni correction). The results are shown in FIGS. 12A-12B.

### Magnetic Resonance Imaging T2 Quantification

MRI analysis was performed at 12- and 24-weeks after CS10 or mixed-cell injection. The transverse relaxation time (T2) mapping of the disc was used as a marker for quantitative assessment for evaluating of collagen matrix, proteoglycan and water content (Belavy et al. PLoS ONE 16(4): e0249855, 2021; Hwang et al., Quantitative Imaging in Medicine and Surgery 6(6): 74455-755, 2016; Lee et al., Magnetic Resonance in Medical Sciences 17(4): 344-49, 2018). Severe disc degeneration would yield low mean T2 value.

As shown in FIGS. 13 and 14, normal L3/4 disc mean T2 in NP was significantly higher than that of degenerated discs such as in CS10 group. The mixed-cell treated groups showed slight structural improvement in nucleus pulposus of L2/3; no improvement was detected in L4/5 disc for both 12- and 24-weeks after cell injection. No change in the mean T2 was observed in AF of all L2/3 and L4/5 discs as compared to normal AF of L3/4 control for both time points of 12 and 24 weeks after cell injection. The results indicated that mixed-cell treatment has a positive structural impact in nucleus pulposus of degenerated lumbar discs.

### Histological Analyses of the Intervertebral Disc

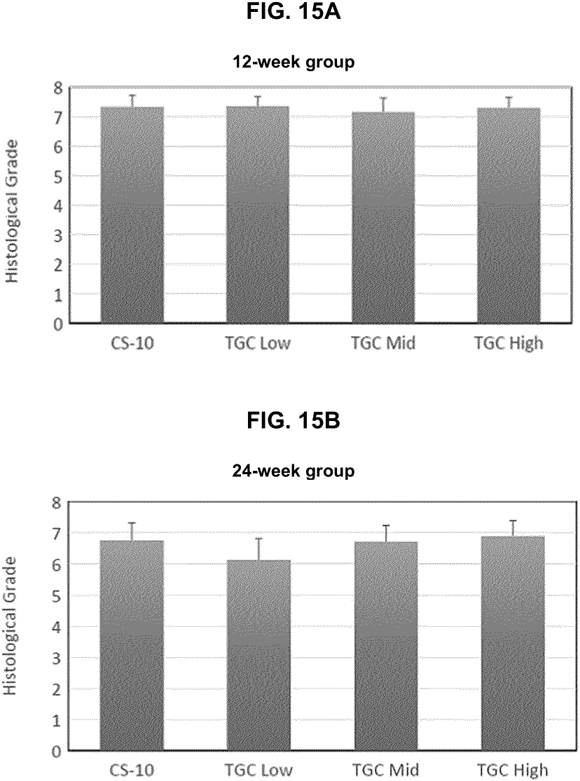

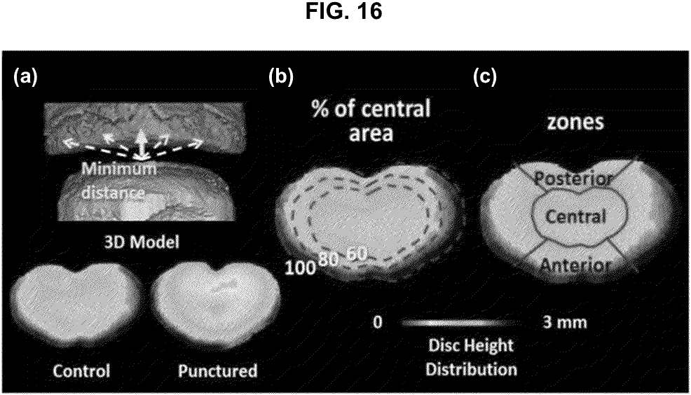

The L2/3 and L4/5 discs of each rabbit were processed for histological analyses. There were no significant changes among the treatment groups at 12 weeks and 24 weeks after injection. The results are shown in FIGS. 15A-15B.

### Micro Ct Analysis of Disc Height Distribution

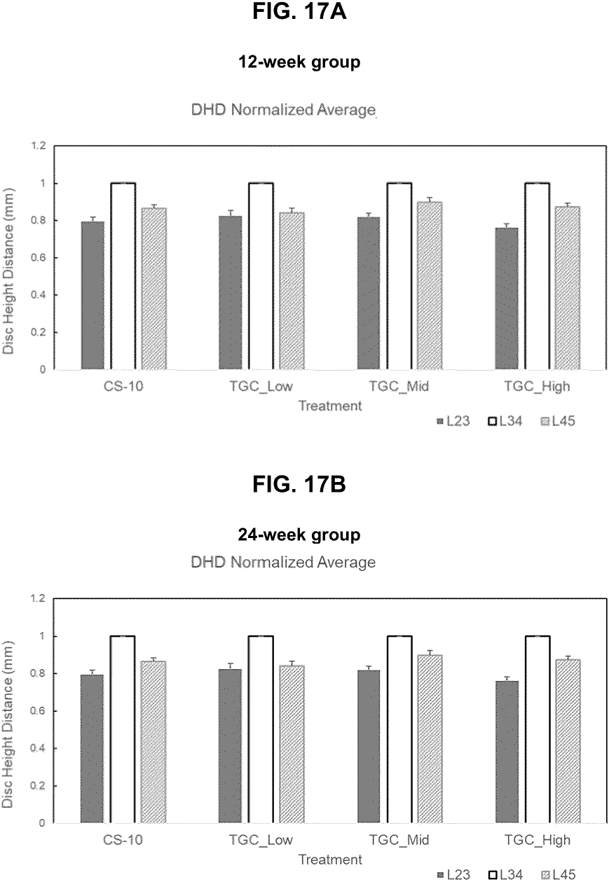

To understand the three-dimension changes in disc height, the specimens taken out from animals were scanned using micro Ct. The data was reconstructed and the surface point cloud data were calculated using mimics, 3D Matic, and custom-designed C++ software as described in the method. The data showed no significant difference among groups at each time point. The results are shown in FIGS. 17A-17B.

Three different TG-C doses ranging from 1.5 × 10⁴ to 1.5 × 10⁵ cells were evaluated in the rabbit anular puncture degenerative disc disease model. All three doses were demonstrated statistically superior to vehicle control group for the disc height index starting from 4 to 24 weeks after TG-C administration. All TG-C treated groups exhibit a statistically significant structural recovery by MRI grading analysis (lower Pfirrmann scores or less degeneration) of L4/5 disc from the 12-week group, whereas minor improvement was observed in L2/3 discs of two high doses of TG-C cells of both 12- and 24-week groups. In addition, MRI T2 quantification results showed a slight improvement with increasing level of TG-C for NP structure after 12 and 24 weeks. Taken together, treating degenerative disc disease induced by puncturing of spinal discs in the rabbit model with TG-C demonstrates significant structural recovery in disc height. The results support TG-C as a potential therapy for treating degenerative disc disease.

What is claimed is:

1. A method for restoring a damaged or degenerating intervertebral disc in a subject in need thereof, comprising administering a composition comprising a mixed cell population to an intervertebral disc site of the subject, wherein the mixed cell population comprises a first mammalian cell comprising an exogenous nucleotide sequence encoding a protein having an intervertebral disc regenerating function and a second mammalian cell that does not comprise the exogenous nucleotide sequence and is a connective tissue cell.

2. The method according to claim 1, wherein the protein having an intervertebral disc regenerating function belongs to the TGF-β superfamily.

3. The method according to claim 2, wherein the TGF-β protein is a human or recombinant TGF-β1 protein.

4. The method according to claim 1, wherein the first mammalian cell is a human embryonic kidney cell or an epithelial cell, and the second mammalian cell is chondrocytes.

5. The method according to claim 4, wherein the human embryonic kidney cell is modified to stably express TGF-β protein.

6. The method according to claim 4, wherein the human embryonic kidney cell or an epithelial cell is irradiated.

7. The method according to claim 4, wherein the chondrocyte is a non-disc chondrocyte or a juvenile chondrocyte.

8. The method according to claim 7, wherein the chondrocyte is a primed chondrocyte.

9. The method according to claim 8, wherein the chondrocyte is primed by incubation with a cytokine.

10. The method according to claim 9, wherein the cytokine is a member of the TGF-β superfamily.

11. The method according to claim 9, wherein the cytokine is TGF-β1 is derived from the first mammalian cell expressing TGF-β1.

12. The method according to claim 1, wherein the first and/or the second mammalian cell is allogeneic relative to the subject.

13. The method according to claim 1, wherein the damaged or degenerating intervertebral disc is a herniated disc or a thinning disc.

14. The method according to claim 13, wherein the mixed cell population contains a plurality of the first mammalian cells and a plurality of the second mammalian cells at a ratio of about 1 to 1-10.

15. The method according to claim 14, wherein the ratio is about 1 to 3.

16. The method according to claim 13, wherein the composition further comprises a cytokine.

17. The method according to claim 16, wherein the cytokine is a member of the TGF-β superfamily.

18. The method according to claim 13, wherein the composition further comprises a pharmaceutical carrier.

19. The method according to claim 18, wherein the pharmaceutical carrier comprises about 10 to 20 % w/w dimethyl sulfoxide and about 1 to 5 % w/w saccharose.

20. The method according to claim 13, wherein the method improves structural recovery by increaseing the disc height index of the damaged or degenerating intervertebral disc as measured by X-ray analysis.

21. The method according to claim 13, wherein the method improves the collagen matrix, proteoglycan or water content of the damaged or degenerating intervertebral disc.

22. The method according to claim 13, wherein the method improves structural recovery at an intervertebral disc defect site as measured by a magnetic resonance imaging analysis of the intervertebral disc defect site.
